(19) United States
(12) Patent Application Publication
(10) Pub. No.: US 2026/0301908 A1
(43) Pub. Date: Oct. 1, 2026

(54) METHODS AND SYSTEMS FOR MANAGING DIABETES

(71) Applicant: Eli Lilly and Company, Indianapolis, IN (US)

(72) Inventors: Emmanuel CHIGUTSA, Zionsville, IN (US); Eyal DASSAU, Acton, MA (US); Michelle Lynne KATZ, Newton, MA (US); Christof KAZDA, Vagnas (FR); Zhao LU, Zionsville, IN (US); Jie XUE, Zionsville, IN (US); Bo ZHANG, Bloomington, IN (US)

(21) Appl. No.: 19/477,673

(22) PCT Filed: May 1, 2024
(86) PCT No.: PCT/US2024/027205

§ 371 Date: Oct. 22, 2025

Related U.S. Application Data
(60) Provisional application No. 63/499,796, filed on May 3, 2023.
(60) Provisional application No. 63/571,570, filed on Mar. 29, 2024.

(51) Int. Cl. G16H 20/17; G16H 50/50

(52) U.S. Cl. CPC G16H 20/17; G16H 50/50

(57) ABSTRACT

This disclosure relates generally to systems, methods and devices used in connection with the management of diseases such as diabetes.

### FIELD

This disclosure relates generally to systems, methods and devices used in connection with the management of diseases such as diabetes. Embodiments include systems, methods and devices for use in connection with long-acting medications, such as insulin receptor agonists, for managing glucose levels in a subject.

### BACKGROUND

Diabetes is a chronic disorder characterized by hyperglycemia resulting from defects in insulin secretion, insulin action, or both. Type 1 diabetes (TID) is characterized by little or no insulin secretory capacity, and people with TID require insulin for survival. Type 2 diabetes (T2D) is characterized by elevated blood glucose levels resulting from impaired insulin secretion, insulin resistance, excessive hepatic glucose output, and/or contributions from all of the above. In many people with T2D, the disease progresses to a requirement for insulin therapy.

Because persons with TID produce little or no insulin, effective insulin therapy generally involves the use of two types of exogenously administered insulin: a rapid-acting, mealtime insulin provided by bolus injections, and a relatively longer-acting, basal insulin, often administered once or twice daily to control blood glucose levels between meals. Treatment of persons with T2D typically begins with prescribed weight loss, exercise, and a diabetic diet, but when these measures fail to control elevated blood sugars, then oral medications and incretin-based therapy may be necessary. When these medications are still insufficient, treatment with insulin is considered. Persons with T2D whose disease has progressed to the point that insulin therapy is required are generally started on a single daily injection of a long-acting, basal insulin.

Some basal insulins currently available include insulin glargine, for example insulin glargines sold under the tradenames BASAGLAR® and LANTUS®, insulin detemir, sold under the tradename LEVEMIR®, and insulin degludec, sold under the tradename TRESIBA®. These insulins are each indicated for once-daily administration. Treatment regimens involving daily injections of existing insulin therapies can be burdensome to administer and can result in undesired side effects, such as low glucose levels or weight gain. Some persons with diabetes (PwD) are unwilling or unable to comply, or are incapable of complying, with the insulin therapy necessary to maintain close control of blood glucose levels.

Insulin products with longer duration of action are under development. Insulin products of these types may require fewer injections than currently available insulin products, including, for example, once-weekly, Such products have the potential to improve acceptance and compliance.

There remains a need for insulin therapies requiring fewer injections than currently available insulin products. The capability of efficaciously providing such insulin therapies, for example either without increasing or by reducing risk of undesired side effects such as hypoglycemia, compared to currently available insulin products would be desirable. Such insulin therapies that can be administered in a manner that allows subjects to relatively quickly reach steady-state serum levels would be particularly desirable. Tools or devices capable of effectively and efficiently providing these needs would be advantageous.

### SUMMARY

This disclosure relates generally to systems, methods and devices used in connection with the management of diseases such as diabetes.

According to an embodiment of the present disclosure, a system comprises a computing system programmed to calculate a final insulin dose using an operation comprising: calculating a proposed insulin dose; iteratively adjusting the proposed insulin dose until a predicted first level is greater than a first threshold; iteratively adjusting the proposed insulin dose until a predicted second level is less than a predetermined maximum threshold; and determining the final insulin dose after the iteratively adjusting steps.

According to another embodiment of the present disclosure, a method comprises calculating a proposed insulin dose, iteratively adjusting the proposed insulin dose until a predicted first level is greater than a first threshold, iteratively adjusting the proposed insulin dose until a predicted second level is less than a predetermined maximum threshold, and determining a final insulin dose after the iteratively adjusting steps.

### BRIEF DESCRIPTION OF THE DRAWINGS

FIG. 1 is a diagrammatic illustration of a diabetes management system in accordance with embodiments.

FIG. 2 is a diagrammatic illustration of the dosing module shown in FIG. 1 in accordance with embodiments.

FIG. 3 is a diagrammatic illustration of an exemplary method implemented by the insulin naïve module shown in FIG. 2 to determine a loading dose for an insulin naïve subject in accordance with embodiments.

FIG. 4 is a diagrammatic illustration of an exemplary method implemented by the basal switch module shown in FIG. 2 to determine a loading dose for a subject switching from daily basal insulin therapies in accordance with embodiments.

FIG. 5 is a diagrammatic illustration of an exemplary method implemented by the safety module shown in FIG. 2 to determine whether to recommend a reduced dose, and the amount of any such reduced dose, in accordance with embodiments.

FIG. 6 is a diagrammatic illustration of an exemplary method for determining the safety concern levels of the method described in connection with FIG. 5, in accordance with embodiments.

FIG. 7 is a diagrammatic illustration of an exemplary method for determining the types and numbers of reduction counts of the method described in connection with FIG. 6, in accordance with embodiments.

FIG. 8 is a table listing criteria defining types of reduction counts in a manner similar to that of the method described in connection with FIG. 7, in accordance with embodiments.

FIG. 9 is a diagrammatic illustration of a method by which the type of dose reduction may be determined in connection with the method of FIG. 5, in accordance with embodiments.

FIG. 10 is a diagrammatic illustration of a method by which the dose reduction amount may be determined in connection with the method of FIG. 5, in accordance with embodiments.

FIG. 11 is a diagrammatic illustration of a method by which the proposed reduced dose may be determined in connection with the method of FIG. 5, in accordance with embodiments.

FIG. 12 is a diagrammatic illustration of a method by which the proposed reduced dose may be verified and/or adjusted in connection with the method of FIG. 5, in accordance with embodiments.

FIG. 13 is a diagrammatic illustration of a method by which the titration module shown in FIG. 2 may determine whether to use the explore module or the exploit module to determine titration doses of insulin, in accordance with embodiments.

FIG. 14 is a diagrammatic illustration of a method by which the titration module shown in FIG. 2 may determine whether to switch or transition from use of the explore module to use of the exploit module to determine titration doses of insulin in connection with the method of FIG. 13, in accordance with embodiments.

FIG. 15 is a diagrammatic illustration of a summary of criteria that can be used by the titration module shown in FIG. 2 to determine whether to transition from use of the explore module to use of the exploit module to determine titration doses of insulin in connection with the method of FIG. 13, in accordance with embodiments.

FIG. 16 is a diagrammatic illustration of embodiments of a method by which the titration module shown in FIG. 2 may determine whether to transition to use of the explore module to determine the titration dose of insulin if the previous dose was determined using the exploit module in connection with the method of FIG. 13.

FIG. 17 is a diagrammatic illustration of a method that can be used by the explore module shown in FIG. 2 to determine titration doses, in accordance with embodiments.

FIG. 18 is a diagrammatic illustration of a method that can be used by the explore module shown in FIG. 2 to determine the proportional parameter Pk in connection with the method shown of FIG. 17, in accordance with embodiments.

FIG. 19 is a diagrammatic illustration of a method that can be used by the explore module to determine whether to recommend a boost dose, and the amount of any such boost dose in connection with the method shown in FIG. 18, in accordance with embodiments.

FIG. 20 is a diagrammatic illustration of a summary of criteria that can be used by the titration module 32 shown in FIG. 2 to determine whether to recommend a boost dose in connection with the method shown in FIG. 18, in accordance with embodiments.

FIG. 21 is a diagrammatic illustration of a method that can be used by the exploit module shown in FIG. 2 to determine titration doses, in accordance with embodiments

FIG. 22 is a diagrammatic illustration of a method that can be used by the exploit module shown in FIG. 2 to verify and/or adjust the proposed initial dose in connection with the method shown in FIG. 21, in accordance with embodiments.

FIG. 23 is a diagrammatic illustration of a method that can be used by the exploit module shown in FIG. 2 to verify and/or adjust the proposed initial dose in connection with the method shown in FIG. 21, in accordance with embodiments.

FIG. 24 is a diagrammatic illustration of a system that can incorporate and be used in connection with the diabetes management system 10 shown in FIG. 1, in accordance with embodiments.

While this disclosure is amenable to various modifications and alternative forms, specific embodiments have been shown by way of example in the drawings and are described in detail below. The intention, however, is not to limit the disclosure to the particular embodiments described but instead is intended to cover all modifications, equivalents, and alternatives falling within the scope of the appended claims.

### DETAILED DESCRIPTION

### Introduction and Long-Acting Insulin Background

Computer-implemented methods and tools for treating or otherwise managing diseases by the determination and provision of efficacious dosing regimens of medication are described herein. The methods and tools are particularly well suited for use in connection with relatively long-acting medications or drugs. These tools and methods are described herein in connection with relatively long-acting insulin receptor agonists suitable for example with once-weekly dosing, as compared for example with commonly used daily basal doses, for the management of diabetes. However, the methods and tools may be used in connection with other suitable medications for the management of other diseases.

When used herein, the terms “insulin” or “insulin receptor agonist” refer to a protein that binds to and activates the insulin receptor, resulting in a lowering of blood glucose levels and/or suppression of hepatic glucose output, characteristics which can be tested and measured using known techniques, such as those shown in the studies described below. The terms “long-acting” “insulin” or “insulin receptor agonist” refers to an insulin receptor agonist having a prolonged pharmacokinetic and/or pharmacodynamic profile to control blood glucose levels between meals when administered no more frequently than once or twice daily. When used herein in connection with an insulin receptor agonist, the term “suitable for once-weekly dosing” refers to a long-acting insulin receptor agonist with a pharmacokinetic and/or pharmacodynamic profile that is sufficiently prolonged to control blood glucose levels between meals when administered no more frequently than once weekly. Examples of such molecules include fusion proteins and are described in U.S. Patent Application Publication 2016/0324932, including insulin efsitora alfa (also referred to herein as Basal Insulin-Fc (BIF)).

Insulin efsitora alfa or BIF is a long-acting basal insulin with a typical half-life of approximately seventeen days, with the associated interindividual variability. Compared with daily basal insulin products, the prolonged half-life enables BIF as a weekly basal injection product that may bring significant convenience and flexibility to patients or other users with diabetes.

BIF comprises a dimer of an insulin receptor agonist fused to a human IgG Fc region, wherein the insulin receptor agonist comprises an insulin B-chain analog fused to an insulin A-chain analog through the use of a first peptide linker and wherein the C-terminal residue of the insulin A-chain analog is directly fused to the N-terminal residue of a second peptide linker, and the C-terminal residue of the second peptide linker is directly fused to the N-terminal residue of the human IgG Fc region. BIF is identified by CAS registry number 2131038-11-2, which provides the following chemical names: (1) Insulin [16-glutamic acid, 25-histidine, 27-glycine, 28-glycine, 29-glycine, 30-glycine] (human B-chain) fusion protein with peptide (synthetic 7-amino acid linker) fusion protein with insulin [47-threonine, 51-aspartic acid, 58-glycine] (human A-chain) fusion protein with peptide (synthetic 20-amino acid linker) fusion protein with immunoglobulin G2 (human Fc fragment), dimer; and (2) Homo sapiens Insulin B-chain [Y16>Y(16), F25>H(25), TPKT27-30>GGGG(27-30)] (1-30) fusion protein with diglycylseryltetraglycyl (31-37) Insulin A-chain [110>T(47), Y14>D(51), N21>G(58)] (38-58) fusion protein with tris(tetraglycylglutaminyl) pentaglycyl (59-78) Homo sapiens Immunoglobulin heavy constant gamma 2 {del-CH1, hinge-(7-12), CH2, CH3[K107>del(300)]} (79-299), dimer 15 (80-80′: 83-83′)-bisdisulfide, expressed in CHO cells, alfa glycosylated.

Each monomer of BIF has the amino acid sequence set forth in SEQ ID NO:1: FVNQHLCGSHLVEALELVCGERGFHYGGGGGGSGGGGGIVEQCCTSTCSL DQLENYCGGGGGQGGGGQGGGGQGGGGGECPPCPAPPVAGPSVFLFPPKP KDTLMISRTPEVTCVVVDVSHEDPEVQFNWYVDGVEVHNAKTKPREEQFN STFRVVSVLTVVHQDWLNGKEYKCKVSNKGLPAPIEKTISKTKGQPREPQ VYTLPPSREEMTKNQVSLTCLVKGFYPSDIAVEWESNGQPENNYKTTPPM LDSDGSFFLYSKLTVDKSRWQQGNVFSCSVMHEALHNHYTQKSLSLSPG (SEQ ID NO:1). Each monomer includes intrachain disulfide bonds between cysteine residues at positions 7 and 44, 19 and 57, 43 and 48, 114 and 174 and 220 and 278. The two monomers are attached by disulfide bonds between the cysteine residues at positions 80 and 83 to form the dimer. BIF's structure, function and production are described in more detail in U.S. Patent Application Publication No. 2016/0324932.

When used herein, the term “BIF” refers to any insulin receptor agonist comprised of two monomers having the amino acid sequence of SEQ ID NO:1, including any protein that is the subject of a regulatory submission seeking approval of an insulin receptor agonist product that relies in whole or part upon data submitted to a regulatory agency by Eli Lilly and Company relating to BIF, regardless of whether the party seeking approval of said product actually identifies the insulin receptor agonist as BIF or uses some other term.

Multiple aspects of dosing regimens for and methods of using long-acting insulin receptor agonist suitable for relatively long-acting (e.g., once-weekly) dosing are described herein. In certain aspects, the regimens and methods described herein include determination and administration of an initial or loading dose of such insulin receptor agonists. In other aspects, the regimens and methods described herein include determination and administration of maintenance or titration doses, including when and how to adjust (e.g., explore) or to generally retain previous (e.g., exploit) titration doses.

The methods and tools described herein provide a controlled, safe environment for titration for insulin naive and basal switch subjects with, for example, type 2 diabetes (T2D). An objective is to bring the fasting blood glucose into a target range within a relatively short period of time (e.g., six to twelve weeks), without additional undesired disease-related risk, such as for example hypoglycemia risk, compared to daily basal insulin.

Ranges and individual levels of parameters including blood glucose and insulin activity levels and time periods, such as for example certain target ranges, ranges that include values that are relatively high with respect to the target ranges, and ranges that include values that are relatively low with respect to the target ranges, are provided in this disclosure for purposes of example only. Ranges of these types may for example be reported in relevant published medical and other literature relating to diabetes and are not intended to be used for or appropriate for any particular subject. Terms relating to “safety” and “risk,” are used in this disclosure only for purposes of relative descriptions in connection with objective bases of safety and/or risk as reported in relevant published medical and other literature relating to diabetes and are not intended to imply any particular amounts of safety and/or risk, e.g., for particular subjects. In particular, blood glucose levels or values in the range of 80-120 mg/dl are used as target values only for example, as are the various levels or values above or below that ranges. Terms such as “safety concern levels” are used only to indicate relative degrees of safety and/or risk of the types known and described in the relevant literature (e.g., in connection with hypoglycemia or hyperglycemia, and in at least some cases compared to those reported in connection with commonly used daily basal insulin), and are not intended to indicate that they are associated with any particular safety or risk level, e.g., for particular subjects.

### Overview

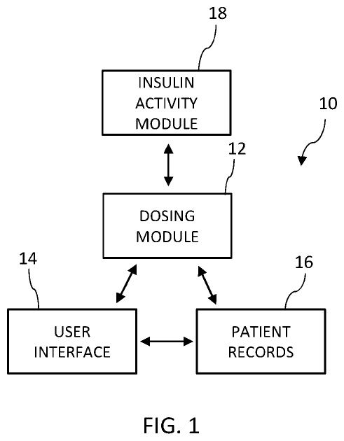

FIG. 1 is a diagrammatic illustration of components of a diabetes (i.e., a type of disease) management system 10 in accordance with embodiments. As shown, system 10 includes dosing module 12, user interface 14, patient (e.g., a user or subject) records 16 and insulin (i.e., a type of medication or drug) activity module 18 (or IA module 18). As described in greater detail below, dosing module 12 defines a method or algorithm providing proposed or recommended doses of drugs or medications, such as long-acting basal insulin, for a particular subject. User interface 14, which as described in greater detail below may include a graphical user interface (GUI) of a mobile device or computing system, is used by a user, such as for example the patient, or a physician or other health care provider (HCP), to access the system 10. For example, the user can input information into the system 10, access dosing module 12 and/or receive medication dose recommendations through the user interface 14. Dosing module 12 determines the proposed doses based on certain parameters or information about the subject, including insulin activity levels that may be determined by insulin activity module 18. Subject information used by the dosing module 12 and insulin activity module 18 may be received through the user interface 14 (e.g., from the patient and/or physician), a glucose sensor, and/or obtained from patient records 16. Patient records 16 may, for example, include one or more electronic medical records (EMR) maintained by the patient's health care providers. Table 1 below lists examples of the types of information used by embodiments of dosing module 12.

TABLE 1

| | |
|---|---|
| Parameter Name | Description |

Basic Information

| | |
|---|---|
| Weight | Patient weight (e.g., in kilogram) |
| HbA1c | Patient HbA1c value in percentage |
| patientType | Identifier of patient type: 0 (insulin naive) and 1 (basal switch) |
| timeStampOfInitialization | Timestamp of patient creation in the system |
| timeStampOfCurrentExecution | Timestamp of the current execution |

Fasting Blood Glucose Information

| | |
|---|---|
| numberOfFastingBloodGlucose | Number of fasting blood glucose records |
| timeStampOfFastingBloodGlucose | Timestamps of the fasting blood glucose records |
| fastingBloodGlucose | Fasting blood glucose values (e.g., in mg/dL) |

Hypoglycemia Event Information

| | |
|---|---|
| numberOfHypoglycemiaEvent | Number of hypoglycemia event records passed to the function |
| timeStampOfHypoglycemiaEvent | Timestamp of each hypoglycemia event |
| bloodGlucoseOfHypoglycemiaEvent | Blood glucose value of the hypoglycemia event |
| isRequiringAssistance | If the hypoglycemia event is labeled as requiring assistance |
| isFlaggedByDoctor | If the hypoglycemia event is flagged by HCP |
| isSymptomatic | If the hypoglycemia event is labeled as symptomatic |
| isNocturnal | If the hypoglycemia event is labeled as nocturnal |

Dose Administration Information

| | |
|---|---|
| numberOfDoseAdministration | Number of dose administration records passed to the function |
| timeStampOfDoseAdministration | Timestamp of dose administration |
| administeredDose | Administered dose amount (e.g., in mg) |
| isLoadingDose | If the administered dose is a loading dose |

Information elements such as the subject's HbA1c (glycated hemoglobin) values and fasting blood glucose (FBG) values may be measured information characterizing physiological parameters of the subject representative of the disease. Other information elements such as those relating to hypoglycemia events are representative of subject events relating to the disease. These subject events may, for example, have been recorded by the subject's health care provider during patient visits, or provided by the subject or HCP though the user interface 14. Blood glucose values measured early in a given day, such as around 8:00 AM, may be considered fasting blood glucose values. In embodiments, for example, the terms “fasting blood glucose,” “FBG” and similar terms may refer to blood sugar levels from a sample of blood taken after a subject fasts for at least eight hours. When used in the context of determining the dose of insulin receptor agonist suitable for once weekly dosing to be administered to a subject, unless otherwise specified herein, the subject's FBG may be determined as the median FBG from multiple days, such as for example at least three days and no more than a predetermined number of days such as seven days.

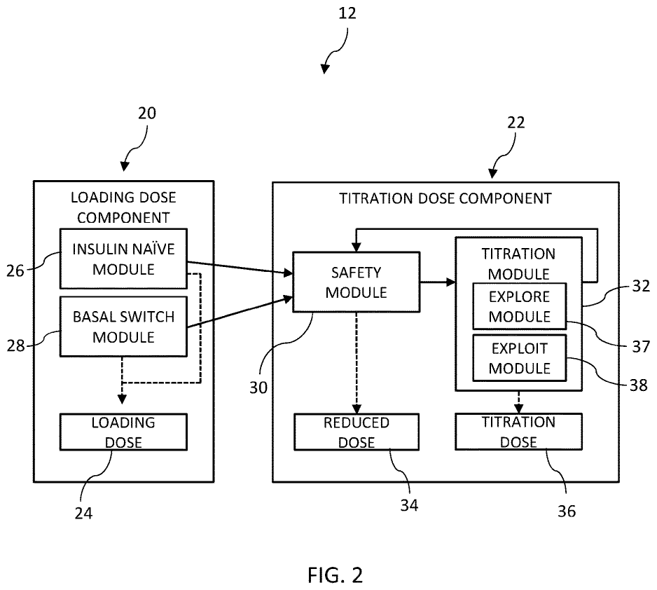

FIG. 2 is a diagrammatic illustration of the dosing module 12 in accordance with embodiments. As shown, the dosing module 12 includes a loading dose component 20 and a titration dose component 22. Loading dose component 20 determines a loading dose 24. Titration dose component 22 determines doses such as reduced doses 34 and titration doses 36 of the medication to be recommended to the subject after the initial, loading dose (e.g., second and subsequent doses) with the objective of maintaining the subject's blood glucose values with the target range. In certain embodiments, the regimens and methods described herein provide for determination and administration of one or more loading doses 24, reduced doses 34 and titration doses 36 to enable subjects to reach target FBG levels after administration of as few doses as possible, and for example while minimizing risks such as hypoglycemia.

When used herein, “dose,” “doses” and similar terms may refer to the quantity of insulin receptor agonist suitable for relatively long periods such as once weekly dosing that is administered to an individual in discrete amount at a particular point in time. When used in connection with the terms dose, dosing, doses and the like, the term “adjustment” refers to the quantity of any decrease or increase to the dose administered during a prior time period, such as for example the prior or preceding week or seven days. When used in connection with the terms dose, dosing, doses and the like, the term “regimen” refers to a set of guidelines for determining and administering one or more doses and/or adjustments thereto.

In embodiments, the loading dose 24 is an initial and typically higher dose (e.g., a first dose) of the medication that may be provided to the subject at the beginning of a course of a treatment regimen to increase the speed and reduce the time needed to cause the medication to produce a desired or target blood glucose level in the subject (e.g., 80 mg/dL-100 mg/dL for some subjects) or insulin activity level. With a long elimination half-life, administration of once-weekly insulin receptor agonists suitable for once weekly dosing following a weekly dosing regimen could result in pharmacokinetic steady-state not being reached for multiple weeks. In addition, the long elimination half-life of such products when administered once-weekly for multiple weeks may result in peak concentration levels significantly greater than the peak serum concentration level that would be observed after administration of a single dose. Certain embodiments of the regimens and methods described herein address these issues through administration of a single dose designed to reduce the time to reach pharmacokinetic steady state. Thus, when used herein, “loading dose” or “one-time starting dose” and similar terms refer to a first dose of relatively long-acting insulin receptor agonist suitable for example, once-weekly dosing administered to a given subject that is larger than (and in embodiments, may be a multiple of) the dose that would be expected to be used for long-term of maintenance treatment. For example, the loading dose may be administered with the purpose of attaining adequate therapeutic concentrations as quickly as possible, instead of waiting for pharmacokinetic steady-state which may otherwise take relatively longer periods of time such as for example several weeks.

In certain embodiments, the loading dose ranges from about 1.5 to about 5 times greater than the expected weekly maintenance dose. In certain embodiments, the loading dose ranges from about 1.5 to about 3 times greater than the expected weekly maintenance dose. In certain embodiments, the loading dose is about 1.5, 1.6, 1.8, 2 or 3 times greater than the expected weekly maintenance dose.

For BIF, steady-state serum concentration levels following initiation of once-weekly dosing is predicted to be reached in approximately 8 to 12 weeks with approximately 3-fold higher concentration due to accumulation than after single dose. In certain preferred embodiments, the insulin receptor agonist suitable for once weekly dosing is BIF, and the loading dose is about 3 times greater than the expected weekly maintenance dose.

In the embodiments shown in FIG. 2, the loading dose component 20 includes an insulin naïve module 26 and a basal switch module 28. The insulin naïve module 26 may be used to determine the loading dose 24 for subjects that have not previously been treated by insulin therapies. The basal switch module may be used to determine the loading dose 24 for subjects that have previously been treated with prior basal insulin therapies, such as shorter-acting therapies for example, and that are switching to a longer-acting insulin therapy such as BIF. System 10 can determine whether to use the insulin naïve module 26 or the basal switch module 28 by any of several different approaches. For example, information retrieved from the patient records 16 or input through the user interface 14 can be analyzed to determine whether the insulin naïve module 26 or the basal switch module 28 is suitable for a particular subject. In other embodiments, the user selects which of the modules 26 or 28 to use (e.g., through the use user interface 14).

Titration dose component 22 determines doses of the medication to be recommended to the subject after the initial, loading dose (e.g., second and subsequent doses) with the objective of maintaining the subject's blood glucose values within the target range. Such maintenance doses determined by the titration dose component 22 are referred to as reduced doses 34 and titration doses 36 in the embodiments shown in FIG. 2. In general, “reduced dose,” “titration dose” and similar terms may refer to any single dose of insulin receptor agonist suitable for once, and for example weekly, dosing other than a loading dose, as defined above.

In the illustrated embodiments, the titration dose component 22 includes a safety module 30 and a titration module 32. As described in greater detail below, by these embodiments the safety module 30 determines whether a reduced dose 34, for example a dose that is less than a previously-determined or previous-administered dose by the titration dose component 22, should be determined and recommended to the subject to maintain the subject's blood glucose levels within the target range. In some embodiments, the reduced doses 34 may be overwritten by a physician. Safety module 30 uses certain elements of the subject information to make the determination of whether to propose a reduced dose 34.

When the safety module 30 determines not to recommend a reduced dose 34, a “regular” titration dose 36 is determined for recommendation by the titration module 32. As shown in FIG. 2, the illustrated embodiment of titration module 32 includes an exploration or explore module 37 and an exploitation or exploit module 38. The titration dose 36 is determined by one of the explore module 37 or the exploit module 38. As described in greater detail below, the explore module 37 generally explores dosing levels to determine optimal levels of insulin in the subject, and the exploit module 38 generally maintains dosing levels to maintain optimal levels of insulin in the subject.

In connection with the description provided herein, a proposed dose being determined for recommendation to a subject may be referred to as a current dose or doseᵢ. The previously determined and recommended dose immediately preceding the current dose, and which may be administered to the subject, may be referred to as a first earlier dose or doseᵢ₋₁. The previously determined dose determined and immediately before the first earlier dose may be referred to as a second earlier dose or doseᵢ₋₂. The period of time between the first earlier dose and the current dose may be referred to as a first earlier dose period, and the period of time between the second earlier dose and the first earlier dose may be referred to as the second earlier dose period. Similar terminology may be used for doses and dose periods before the second earlier dose and the second earlier dose period. For certain insulins such as the BIF insulin described above, the length of a dose period is seven days.

### Loading Dose Component

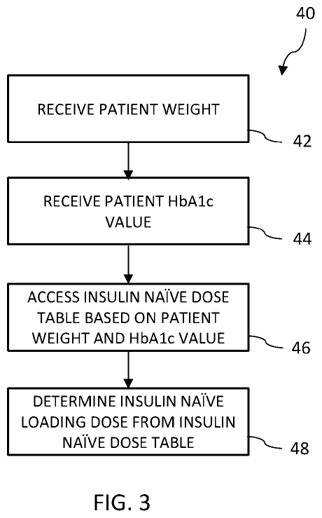

FIG. 3 is a diagrammatic illustration of a method 40 that can be implemented by the insulin naïve module 26 to determine a loading dose 24 for an insulin naïve subject in accordance with embodiments. As shown by steps 42 and 44, respectively, the illustrated embodiments of method 40 receive and use certain parameters such as the subject's weight and HbA1c values as a basis for determining the loading dose 24 for an insulin naïve subject. The parameters may be received, for example, by accessing the patient's records 16. As indicated by step 46, method 40 also makes use of a stored insulin naïve table that defines a range of insulin naïve loading dose values based on ranges of the weights and HbA1c values (e.g., a lookup table). In embodiments, for example, the rows of the table accessed by step 46 may be indexed by HbA1c values, ranging from 6% to 12% using 0.1% increments. For HbA1c values between 12% and 15%, it is treated as 12% in embodiments. Columns of the table may be indexed by weight, for example ranging from 50 kg to 200 kg in 1 kg increments. The dose value accessed from the stored insulin naïve table at step 46 may then be provided as the proposed or recommended insulin naïve loading dose 24 as shown by step 48. Other embodiments of insulin naïve module 26 use other algorithms and approaches for determining a loading dose of the medication for an insulin naïve subject.

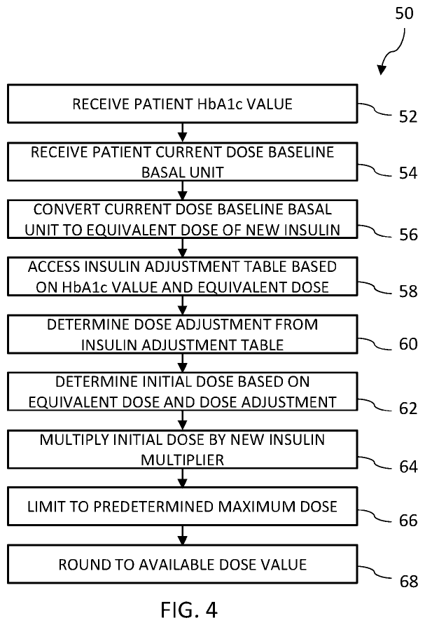

FIG. 4 is a diagrammatic illustration of a method 50 that can be implemented by the basal switch module 28 to determine a loading dose 24 for a subject switching from shorter-acting insulin therapies in accordance with embodiments. As shown by steps 52 and 54, respectively, the illustrated embodiments of method 50 receive and use certain parameters such as the subject's HbA1c value and the baseline basal unit of their current medication (e.g., short-acting insulin or daily basal insulin) as a basis for determining the loading dose 24 for a basal switch subject. The parameters may be received, for example, by accessing the patient's records 16. Because the International Units (IU) level of the subject's then-current insulin dose (e.g., a measure of the biological activity of the insulin) may be different than the IU of the new longer-acting insulin, the current insulin dose baseline basal unit is converted to the equivalent dose for the new insulin at step 56, as needed. For example, when converting some relatively short-acting insulin doses of fifty IU to an equivalent long-acting BIF dose of ten IU, the baseline basal unit of the current insulin may be divided by a factor of five.

As indicated by step 58, method 50 also makes use of a stored insulin adjustment table that defines a range of adjustment values based on ranges of parameters such as HbA1c and new insulin equivalent dose values (e.g., a lookup table). In embodiments, for example, the rows of the table accessed at step 58 may be indexed by ranges of HbA1c values (e.g., 6%≤HbA1c<6.6%; 6.6%≤HbA1c<7%), and the columns of the table may be indexed by ranges of equivalent dose values (e.g., baseline basal≤15 IU; 15 IU<baseline basal≤30 IU). The adjustment values may be characterized as amounts of changes (e.g., decreases, increases, or no changes), in mg. Generally, in some embodiments, at relatively low HbA1c and equivalent baseline basal dose values, the adjustment values will be decreases. In such embodiments the adjustment values may increase to no change, and to increasing larger increases, with increasing HbA1c and equivalent baseline basal dose values. The insulin adjustment table is accessed at step 58 based on the subject's HbA1c and current equivalent new insulin baseline basal value to determine an appropriate adjustment value, as shown by step 60.

At step 62 method 50 determines an initial loading dose based on the equivalent new insulin dose determined at step 56 and the adjustment value determined at step 60. In embodiments, for example, the initial dose of the new insulin can be determined at step 62 by reducing the equivalent new insulin dose by the dose adjustment value. The illustrated embodiments of method 50 increase the initial loading dose by a factor, such as for example a new insulin multiplier, as shown by step 64. In embodiments the new insulin multiplier may be a stored, predetermined value, such as three. The dose result determined at step 64 may be limited or capped at a stored, predetermined maximum value, such as for example 36.0 mg, as shown at step 66. To account for any quantization of available dose quantities of the new insulin, the dose result determined by the method 50 through step 66 may be rounded to such available dose quantities. For example, at step 68 the dose result may be rounded to the nearest 0.25 mg if it is less than some predetermined value such as 5.0 mg. Additionally or alternatively, if the dose result is greater than or equal to the predetermine value such as 5.0 mg, that dose result may be rounded to the nearest 0.5 mg. The dose value determined at step 68 may then be provided as the recommended insulin basal switch loading dose 24. Other embodiments of basal switch module 28 use other algorithms and approaches for determining a loading dose 24 of the new medication for a subject switching insulin therapies. Although doses in units of mg are described in this disclosure, other embodiments may characterize doses in units of IU (insulin units).

### Insulin Activity Module

Insulin activity is an estimated effect of insulin on lowering the glucose of a subject and may be based, for example, on the pharmacokinetics/pharmacodynamics (PK/PD) and duration of action of the insulin in a subject. Referring back to FIG. 2, titration dose component 22 of the dosing module 12 uses the insulin activity module 18 to determine the current insulin activity (IA) levels or effect of insulin dosing histories on subjects, and to predict future insulin activity (IA) levels or effects of proposed insulin dosing regimens on subjects. Accordingly, the determined insulin activity level provides a measure of the estimated insulin effect that given dosing histories or regimens have or may produce in a subject. Embodiments of the insulin activity module 18 make use of a compartmental model based on the ordinary differential equations 3.1.1-3.1.3 below:

$$\frac{dA}{dt} = - {k}_{1} A \qquad Eq . 3.1 \text{.1}$$

$$\frac{dB}{dt} = {k}_{1} A - {k}_{2} B \qquad Eq . 3.1 \text{.2}$$

$$\frac{dC}{dt} = {k}_{2} B \qquad Eq . 3.1 \text{.3}$$

Time period t may be in the unit of one-thousand, four-hundred and forty (1440) minutes (one day) in embodiments. By this model, a drug such as insulin is administered into compartment A and transferred into compartment B at a linear rate k₁. During the same time period, compartment B empties the drug into compartment C at a linear rate k₂. The insulin activity level is monitored at compartment B. Given initial conditions A(t₀)=A₀, B(t₀)=B₀, and C(t₀)=C₀, solutions to the differential equation system represented by Eqs. 3.1.1-3.1.3 may be represented by Eqs. 3.2.1-3.2.3 below:

$$A ( t ) = b ( {k}_{1} - {k}_{2} ) {e}^{- {k}_{1} t} \qquad Eq . 3.2 \text{.1}$$

$$B ( t ) = - {bk}_{1} {e}_{- k 1 t} - {ce}_{- k 2 t} \qquad Eq . 3.2 \text{.2}$$

$$C ( t ) = a + {k}_{2} {be}_{- k 1 t} + {ce}_{- k 2 t} \qquad Eq . 3.2 \text{.3}$$

$$\text{where:} a = {A}_{0} + {B}_{0} + {C}_{0} , b = \frac{{A}_{0}}{{k}_{1} - {k}_{2}} {e}^{{k}_{1} {t}_{o}} , c = [ \frac{{k}_{1} {A}_{0}}{{k}_{1} - {k}_{2}} + {B}_{0} ] {e}^{{k}_{2} {t}_{o}}$$

As described in greater detail below, titration dose component 22 defines a sequence of N doses {aᵢ} administered at times {tᵢ}, i=1, . . . , N, and the current dose calculation time t_{N+1}. The insulin activity module 18 may compute a complete historical insulin activity level up to t_{N+1}, with a snapshot of the differential equation system taken periodically, such as for example every one-hundred and forty-four (144) minutes. For t∈[t₁,t₂], the insulin activity level may be computed by solving Eqs. 3.1.1-3.1.3 with initial conditions 3.3.1-3.3.3 below:

$$A ( {t}_{1} ) = {a}_{1} \qquad Eq . 3.3 \text{.1}$$

$$B ( {t}_{1} ) = 0 \qquad Eq . 3.3 \text{.2}$$

$$C ( {t}_{1} ) = 0 \qquad Eq . 3.3 \text{.3}$$

For subsequent intervals [t₁,tᵢ₊₁], where 2≤i≤N, the dose amount may be first applied to compartment A as represented by Eq. 3.4 below:

$$A ( {t}_{i} ) ← A ( {t}_{i} ) + {a}_{i} \qquad Eq . 3.4$$

Differential equations 3.1.1-3.1.3 may then be solved with the updated initial conditions. By this approach the insulin activity module 18 may determine a current insulin activity of the subject (e.g., the insulin activity at the time a dose is being determined).

As noted above, the insulin activity module 18 may also be used to predict future insulin activity levels in a subject, such as for example to evaluate and verify the impact or effect of a proposed dose recommendation on glucose lowering in a subject. When the insulin activity module 18 is being used for predictive purposes, the insulin activity level at the current dose calculation time (N+1 may be defined by conditions A (t_{N}+1)=a_{N}+1, B(t_{N}+1)=b_{N}+1, and C(t_{N}+1)=c_{N}+1, and the proposed dose amount is a, scheduled to be administered M times at times t_{N+1}, t_{N+2}, . . . , t_{N+M} in the future. The future insulin activity level in the interval [t_{N+i},t_{N+i+1}], where 1≤i≤M−1 may then be computed by first applying the proposed dose a to compartment A as indicated by Eq. 3.5 below, and then solving the differential equations 3.1.1-3.1.3 with the updated initial conditions.

$$A ( {t}_{N + i} ) ← A ( {t}_{N + i} ) + a \qquad Eq . 3.5$$

Yet another use of the insulin activity module 18 may be the computation or other determination of a relationship between the provision of given, same dose of the drug to a subject on a periodic basis, such as weekly, and the associated median insulin activity level at steady state. As an example, a table (e.g., a lookup table) defining a range of insulin dose amounts and associated median insulin activities can be generated by solving Eqs. 3.1.1-3.1.3 for dose amounts a, where the corresponding insulin activity level is k₁=0.45, k₂=0.045, and applying dose a on the weekly basis for a predetermined number of weeks such as twenty-six weeks. Table 2 below is a diagrammatic illustration of a dose—insulin activity (IA) table in accordance with embodiments. Other embodiments may use a dose—insulin activity table determined by other methods, and/or other approaches for defining the relationship between dose and insulin activity.

TABLE 2
Dose - Insulin Activity

| Dose (mg) | Median IA |
|---|---|
| 0.25 | 0.745977 |
| 0.5 | 1.491954 |
| 0.75 | 2.237931 |
| . . . | . . . |
| 29.5 | 88.2528 |
| 30 | 89.51723 |

The embodiments of the insulin activity module 18 described above effectively implements a population-based pharmacodynamic model that provides estimates of the insulin activity levels that given dosing histories or regimens have or may produce in a subject. Other embodiments of dosing module 12 may use other known or otherwise available models or approaches for determining the activity levels of the medication doses.

### Safety Module

Referring back to FIG. 2, safety module 30 is used to determine whether one or more safety concern levels may be present in connection with determinations of titration doses of the medication during the use of the titration dose component 22. The safety concern levels may, for example, be indications of one or more diabetes conditions determined from objective information in the patient records 16 and based on objective and established medical practices such as those available in published literature. If one or more safety concern levels is identified by the safety module 30, a reduced dose 34 is determined and recommended by the titration dose component 22. If safety concern levels are not identified, titration dose component 22 uses the titration module 32 to determine the titration dose 36. In effect, by these embodiments, the safety module 30 is used to determine whether a reduced dose 34, instead of or as an alternative to a titration dose 36, should be recommended for each dose following the determination of the loading dose 24.

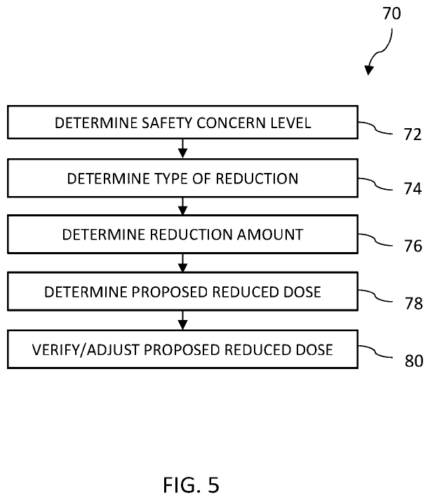

FIG. 5 is a diagrammatic illustration of a method 70 that can be used by the safety module 30 to determine whether to recommend a reduced dose 34, and to determine the amount of any such reduced dose. As shown, at step 72 the method 70 determines a safety concern level. In embodiments, and as described in greater detail below, the safety concern level determined at step 72 may be based on one or more of (1) one or more measured physiological parameters of the subject, such as FBG levels, and/or (2) one or more attributes evidencing or otherwise relating to subject events indicating or relating to the disease, such as hypoglycemia events. A type of dose reduction is determined at step 74. In embodiments and as described in greater detail below, the type of reduction at step 74 may be determined based on factors that are the same as or similar to those used to determine the safety concern level at step 74. A proposed reduced dose is determined at step 78. The proposed reduced dose may then be verified, and optionally adjusted before being verified, at step 80. The verified reduced dose determined by the method 70 may then be provided or recommended as the reduced dose 34.

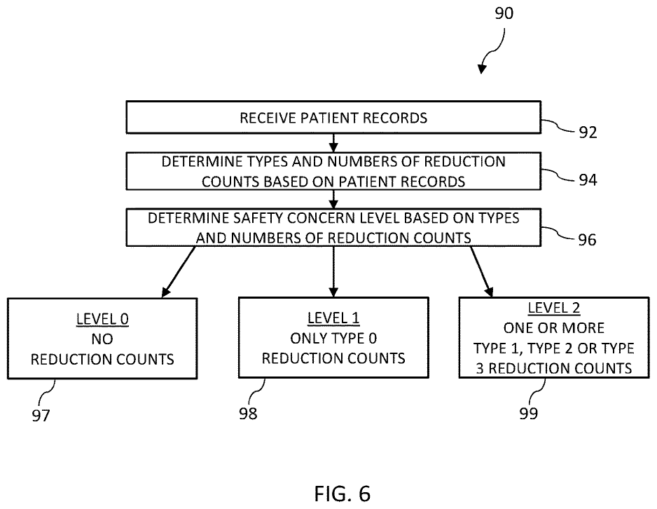

FIG. 6 is a diagrammatic illustration of a method 90 by which the safety concern levels may be determined at step 72 of method 70 (FIG. 5) in embodiments. Method 90 determines the safety concern levels based on subject-specific information relating to and characteristic of the disease in the subject. In embodiments, for example, the subject-specific and disease-related information may include information relating to one or more hypoglycemia events such as (1) one or more measured physiological parameters representative of the disease state, such as for example, blood glucose levels, and/or (2) instances or attributes evidencing disease-related events that occurred with respect to the subject. Disease-related subject events may include, for example instances or indications of hypoglycemia in the subject. As indicated by step 92, the subject-specific disease-related information may be determined from the patient records 16 (shown in FIG. 1). In embodiments, only disease-related information that occurred within a predetermined period of time of the safety concern level determination is used in connection with the determination by method 90.

As shown by step 94, in embodiments the method 90 determines the safety concern level by evaluating the subject-specific disease-related information and identifying different types or states of indications or conditions represented by that information, and the number of instances of those identified types or states of indications. As described below, in embodiments only certain temporal disease-related information (e.g., that occurred within a predetermined period of time of the determination), is used for the determination at step 94. In embodiments, at step 94 the method 90 characterizes the relevant information instances of the disease-related information as being one of a plurality of different types of reduction counts. In the exemplary embodiments described herein the instances are categorized as one of Type 0, Type 1, Type 2 or Type 3, and the number of each such Type 0-Type 3 instances are determined.

At step 96, method 90 determines the safety concern level based on the number and types of the determined reduction counts. In the exemplary embodiments described herein, method 90 characterizes the safety concern level as being one of a Level 0 (step 97), Level 1 (step 98) or Level 2 (step 99). As described in greater detail below, both Level 0 and Level 1 will transfer control to the titration module 32 (FIG. 2). A Level 1 concern will impose no dose increase constraint on the titration module 32, while a Level 0 concern will not impose that additional constraint. A Level 2 concern will lead to a reduced dose directly.

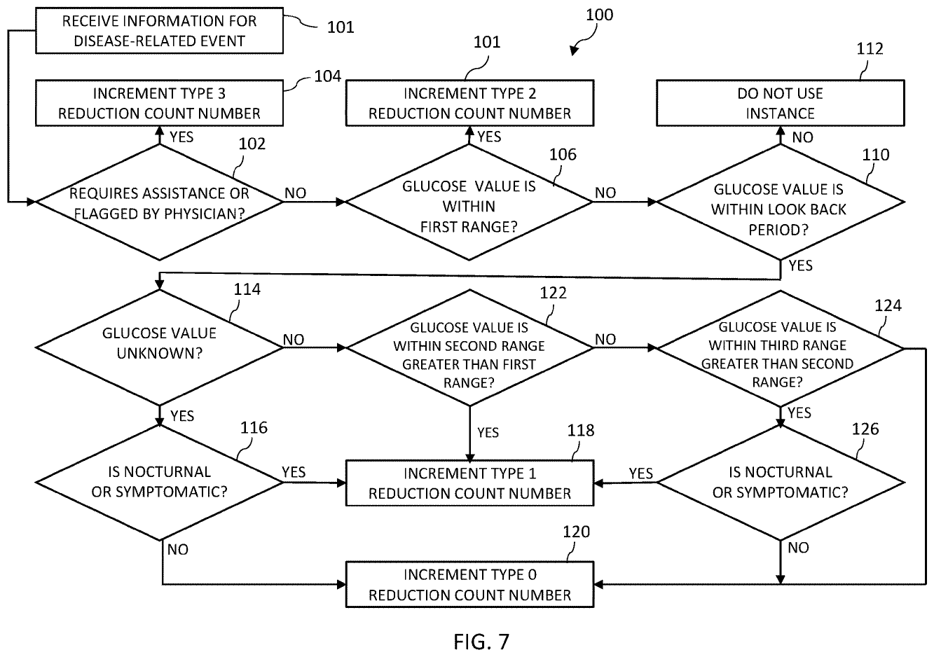

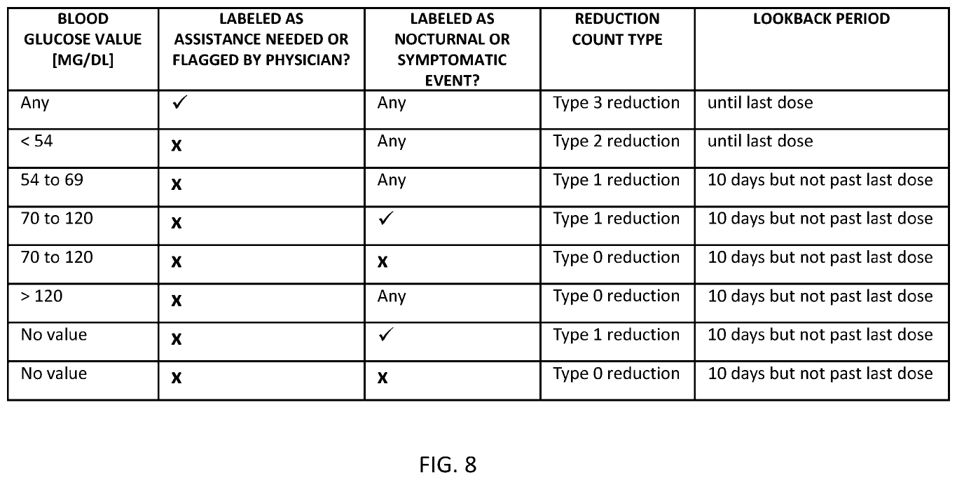

FIG. 7 is a diagrammatic illustration of embodiments of a method 100 that may be used to determine the types and numbers of reduction counts based on the disease-related information instances (e.g., step 94 of FIG. 6). As indicated by step 101, method 100 is based on information in the patient records 16 (FIG. 1), and in embodiments, information relating to measured blood glucose levels and instances of hypoglycemic events in the records. In embodiments, the method 100 is based on information relating to disease-related information instances that occurred (e.g., have associated dates) after a last recommended or administered dose of the insulin (but as described in connection with FIG. 8, for example, in some instances no further back in time than a predetermined look back period such as ten days). For each such instance, method 100 determines at step 102 whether the instance was associated with an event where the subject required assistance or was otherwise flagged or noted by a physician. If a Yes determination is made at step 102, the instance is determined to be a Type 3 instance, and the Type 3 reduction count is incremented as shown by step 104. If a No determination is made at step 102, method 100 determines at step 106 whether the instance is associated with a measured blood glucose value within a first range (e.g., 1 mg/dl-53 mg/dL). If a Yes determination is made at step 106, the instance is determined to be a Type 2 instance, and the Type 2 reduction count is incremented as shown at step 108. If a No determination is made at step 106, method 100 determines at step 110 whether the instance occurred within a predetermined look back period such as ten days. If a No determination is made at step 110, method 100 does not use the instance (e.g., it is not counted as a Type 0-Type 3 instance). If a Yes determination is made at step 110, method 100 continues to evaluate the instance at step 114.

At step 114, method 100 determines whether the instance has an unknown associated blood glucose value. If a Yes determination is made at step 114 (e.g., there is no associated blood glucose measurement), method 100 determines at step 116 whether the instance is labeled as a nocturnal or symptomatic event. If a Yes determination is made at step 116, the instance is determined to be a Type 1 instance, and the Type 1 reduction count is incremented as shown at step 118. If a No determination is made at step 116, the instance is determined to be a Type 0 instance, and the Type 0 reduction count is incremented as shown at step 120.

If at step 114 a No determination is made (e.g., there is an associated blood glucose measurement), the method 100 determines at step 122 whether the instance is associated with a measured blood glucose value within a second range that is greater than the first range (e.g., 54 mg/dL-69 mg/dL continuing with the example above). If a Yes determination is made at step 122, the instance is determined to be a Type 1 instance, and the Type 1 reduction count is incremented as shown at step 118.

If at step 122 a No determination is made, the method 100 determines at step 124 whether the instance is associated with a measured blood glucose value within a third range that is greater than the second range (e.g., 70 mg/dL-120 mg/dL continuing with the example above). If a No determination is made at step 124, the instance is determined to be a Type 0 instance, and the Type 0 reduction count is incremented as shown at step 120.

If a Yes determination is made at step 124, method 100 determines at step 126 whether the instance is labeled as a nocturnal or symptomatic event. If a Yes determination is made at step 126, the instance is determined to be a Type 1 instance, and the Type 1 reduction count is incremented as shown at step 118. If a No determination is made at step 126, the instance is determined to be a Type 0 instance, and the Type 0 reduction count is incremented as shown at step 120.

FIG. 8 is a table listing criteria for defining instances of subject-specific disease information as Type 0, Type 1, Type 2 or Type 3 instances in a manner similar to that by which method 100 determines whether the instances are Type 0-Type 3 instances. Other embodiments use other methods or approaches for characterizing the nature of the instances of the subject-specific disease information.

Referring back to FIG. 6, method 90 determines the safety concern level for the subject using one or both of the types of the instances determined by method 100 and/or the reduction counts (e.g., numbers) of the determined types of the instances. In the illustrated embodiments, method 90 defines the safety level concern as a:
Level 0 Concern-if there are no reduction counts of any of the Type 0-Type 3 instances (e.g., no Type 0-Type 3 instances were identified);
Level 1 Concern-if there are only Type 0 reduction counts (e.g., if there are Type 0 reduction counts, and no Type 1-Type 3 instances were identified);
Level 2 Concern-if one or more reduction counts for any of the Type 1-Type 3 instances (e.g., if any Type 1, Type 2 or Type 3 instances were identified).

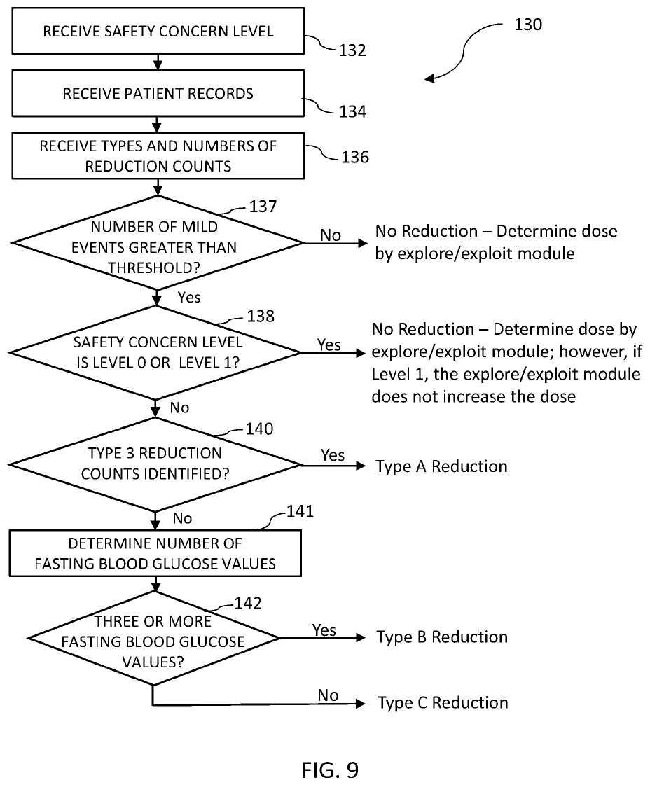

FIG. 9 is a diagrammatic illustration of a method 130 that may be used to determine a type of dose reduction in connection with step 74 in FIG. 5. In the illustrated embodiments, method 130 defines a dose reduction type as one of a plurality of different types. The dose reduction type may be based on factors including, for example, one or more of the safety concerns levels (e.g., as determined by method 90), the types and numbers of the reduction counts (e.g., as determined by method 100), and the number and values of blood glucose measurements such as the FBG values. The type of dose reduction may be determined by other methods or approaches in other embodiments.

As shown in FIG. 9, method 130 receives the safety concern level at step 132, receives the patient records at step 134, and receives the types and numbers of reduction counts at step 136. At step 137, method 130 determines whether the subject has experienced a number of mild events that is greater than a threshold (e.g., 2 or 3 mild daytime hypo events). If the subject has only experienced 1 or 2 events, the safety module 30 (FIG. 2) is not utilized and a titration dose 36 can be determined by the titration module 32. If the subject experienced more than the threshold number of events, the safety module 30 can be used.

If the subject has experienced an event and the titration module 30 (e.g., the explore module 37) is used to determine a titration dose 36, certain boundaries can be placed on the titration module 30 to help prevent excessive dose increments. The boundaries include: (1) that the next dose must be equal to or less than the previous dose amount, (2) the next dose utilizes an additional safety check of a 1-week prediction, and either (3a) if only 1 daytime hypo event occurred, use Equation 3.6 below or (3b) if 2 daytime hypo events occurred, use Equation 3.7 below. The specific values listed in the Equations below are just examples and can be adjusted or otherwise customized.

$$maxIA \times ( 1 + \frac{0.05}{1 + {e}^{- 0.2 \cdot ( medianFbg - 110 )}} ) \qquad Eq . 3.6$$

$$maxIA \times ( 1 + \frac{0.025}{1 + {e}^{- 0.2 \cdot ( mediunFbg - 110 )}} ) \qquad Eq . 3.7$$

At step 138, the received safety concern level is read and reviewed to determine if it is a Level 0 or a Level 1. If a Yes determination is made at step 138, method 130 determines that no dose reduction is recommended. In effect, by step 138, the safety module 30 (FIG. 2) determines that a reduced dose 34 of the medication is not recommended (e.g., may not be needed), and a titration dose 36 can be determined by the titration module 32. As shown in FIG. 9 at step 138, if the safety concern level is determined to be a Level 1, no increase in the dose is permitted by the titration module 32.

If a No determination is made at step 138, method 130 determines at step 140 whether there are one or more Type 3 reduction counts (e.g., whether a Type 3 instance was identified). It a Yes determination is made at step 140, method 130 characterizes or defines the type of reduction as a Type A Reduction.

If a No determination is made at step 140 (e.g., there are no Type 3 reduction counts), method 130 determines at step 141 how many fasting blood glucose records or measurements are available in the patent records 16 within a predetermined period, such as since the previously recommended or administered dose. In embodiments, the predetermined period may have a maximum length, such as for example seven days. At step 142 method 130 determines whether the number of blood glucose records or measurements is a predetermined threshold, such as for example three measurements, four measurements, or more than four measurements. If a Yes determination is made at step 142, method 130 characterizes or defines the type of reduction as a Type B Reduction. If at step 142 less than the predetermined number of fasting blood glucose measurements are available, such as zero, one or two records continuing with the example above, method 130 characterizes or defines the type of reduction as a Type C Reduction.

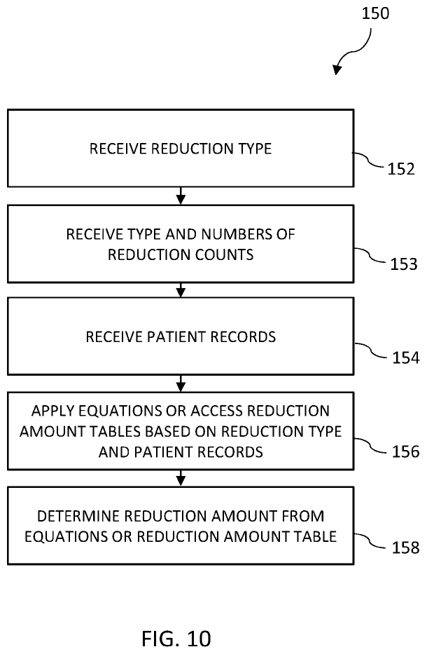

FIG. 10 is a diagrammatic illustration of a method 150 that may be used to determine a dose reduction amount in connection with step 76 in FIG. 5. In the illustrated embodiments, method 150 determines the dose reduction amount in terms of a target reduction in the subject's insulin activity (IA). In other embodiments the dose reduction amount is defined in other terms, such as dose quantity (e.g., in mg). The dose reduction amount may be based on factors including the factors used by method 130 to determine the dose reduction type, one or more measured glucose values such as the FBG, and the types and numbers of the reduction counts.

As shown in FIG. 10, method 150 receives the reduction type at step 152 (e.g., as determined by method 130), the types and numbers of reduction counts at step 153 (e.g., as determined by method 100) and patient records at step 154. As indicated by step 156, method 150 makes use of (1) one or more equations and/or (2) one or more stored reduction amount tables that define the reduction amounts based on the reduction type and information in the patient records 16 such as the fasting blood glucose values.

When a Type 2 reduction count is positive or any Type 1 reduction count is caused by a nocturnal hypoglycemia event, the reduction target is set to −20% if there is 0, 1, or 2 fasting blood glucose records in the previous dosing cycle. Otherwise, the reduction target is given by:

$$\{ \begin{matrix}- 0.2 \; 5 & median FBG < 70 \\ \frac{median FBG - 145}{300} & 70 \leq median FBG \leq 130 \\ - 0.05 & median FBG > 130\end{matrix} \qquad Eq . 3.8 \text{.1}$$

When a Type 1 reduction count is 4, the reduction target is set to −15% if there is 0, 1, or 2 fasting blood glucose records in the previous dosing cycle. Otherwise, the reduction target is given by:

$$\{ \begin{matrix}- 0.2 & median FBG < 70 \\ \frac{3 \times median FBG - 410}{1000} & 70 \leq median FBG \leq 120 \\ - 0.05 & median FBG > 120\end{matrix} \qquad Eq . 3.8 \text{.2}$$

When a Type 1 reduction count is 3, the reduction target is set to −10% if there is 0, 1, or 2 fasting blood glucose records in the previous dosing cycle. Otherwise, the reduction target is given by:

$$\{ \begin{matrix}- 0.2 \; 5 & median FBG < 70 \\ \frac{median FBG - 130}{400} & 70 \leq median FBG \leq 110 \\ - 0.05 & median FBG > 110\end{matrix} \qquad Eq . 3.8 \text{.3}$$

Tables 3, 4 and 5 below are examples of Type A, Type B and Type C reduction amount tables, respectively, in accordance with certain embodiments. For example, Table 3 can be used in situations involving a Type 3 reduction count while the equations noted above can be used for the remaining types of situations. Use of the equations can result in smoother or less drastic changes in dosage amounts.

TABLE 3
Type A Reduction Table

| Type 3 | Reduction | |
|---|---|---|
| Reduction Count | Amount | Additional Requirement |
| Any | −30% IA | Immediate 20% peak IA |
| | | reduction in one week |
| | | prediction |

TABLE 4
Type B Reduction Table

| Type 1 | Type 2 | Median FBG | Reduction |
|---|---|---|---|
| Reduction Count | Reduction Count | (mg/dL) | Amount |
| 1 | 0 | >120 | −5% IA |
| 1 | 0 | 90-120 | −10% IA |
| 1 | 0 | 80-90 | −15% IA |
| 1 | 0 | <80 | −20% IA |
| 2 | 0 | >90 | −15% IA |
| 2 | 0 | 80-90 | −20% IA |
| 2 | 0 | <80 | −25% IA |
| ≥3 | 0 | >90 | −20% IA |
| ≥3 | 0 | 80-90 | −25% IA |
| ≥3 | 0 | <80 | −30% IA |
| Any | >0 | >90 | −20% IA |
| Any | >0 | 80-90 | −25% IA |
| Any | >0 | <80 | −30% IA |

TABLE 5
Type C Reduction Table

| Type 1 | Type 2 | Reduction |
|---|---|---|
| Reduction Count | Reduction Count | Amount |
| 1 | 0 | −15% IA |
| 2 | 0 | −20% IA |
| ≥3 | 0 | −25% IA |
| Any | >0 | −25% IA |

In the embodiments illustrated by Table 3, the reduction amount for all Type A reductions is a predetermined amount, shown for example as a 30% IA reduction. In the embodiments illustrated by Table 4, the reduction amount for Type B reductions is an amount from a range of amounts, such as for example 5% IA-30% IA, and is based on factors such the number of Type 1 reduction counts and median values of the measured glucose values. The median glucose values can be calculated from the values in the patient records 16. In the embodiments shown in Table 4, the range of the reduction amounts is a range of amounts less than the reduction amount in Table 3. In the embodiments illustrated by Table 5, the reduction amount for Type C reductions is an amount from a range of amounts, such as 15% IA-25% IA, and is based on factors such as the number of Type 1 reduction counts. In the embodiments shown in Table 5, the range of the reduction amounts is a range that is within, but smaller than, the range of reduction amounts in Table 4. Other embodiments determine reduction amounts by other approaches and/or using other factors.

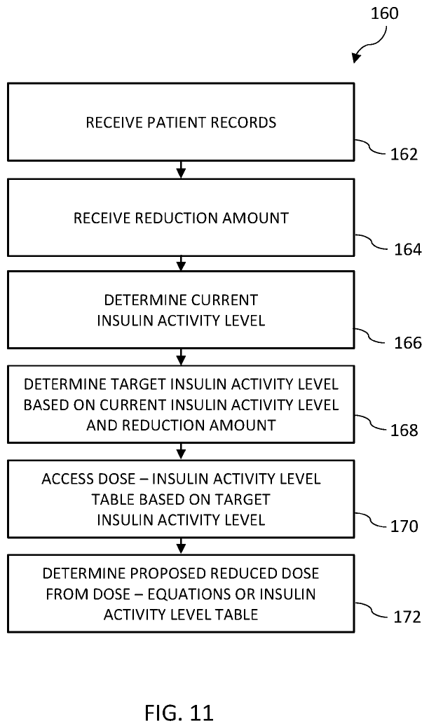

FIG. 11 is a diagrammatic illustration of a method 160 that may be used to determine the proposed reduced dose in connection with step 78 in FIG. 5. In the illustrated embodiments, method 160 receives the patient records at step 162 and the reduction amount (e.g., as determined by method 150). The subject's then-current insulin activity level is computed at step 166, for example by the insulin activity module 18 (as shown in FIG. 1) using information such as the dosing history in the patient records 16. A target insulin activity level is then determined based on the current insulin activity level and the desired reduction amount, as shown by step 168. The determination at step 168 can be made, for example, by subtracting the desired reduction amount from the current insulin activity level. As indicated by step 170, method 160 makes use of a stored dose—insulin activity level table correlating stable doses of the insulin to associated median insulin activity levels. In embodiments, for example, the dose—insulin activity level table may be a table such as the Table 2 described above.

At step 170 the dose—insulin activity table is accessed based on the target insulin activity level determined at step 168. A proposed reduced insulin dose is then determined from the dose—insulin activity table at step 172. The proposed dose determined at step 172 may, in embodiments, be compared to a last recommended or administered dose, and reduced by an amount to cause the reduced dose to be a dose that is smaller than the last administered dose. For example, the reduced dose determined at step 172 may be reduced by an amount that caused the recommended reduced dose to represent a predetermined minimum reduction from the last administered dose. If the last administered dose is a loading dose, the comparison may account for the loading dose multiple (e.g., continuing with the example above, the comparison is made to one-third of the loading dose). Other embodiments determine proposed reduced doses by other approaches, such as for example by inputting information such as the target insulin activity level into a model including an equation defining a continuous relationship between the input and a proposed dose.

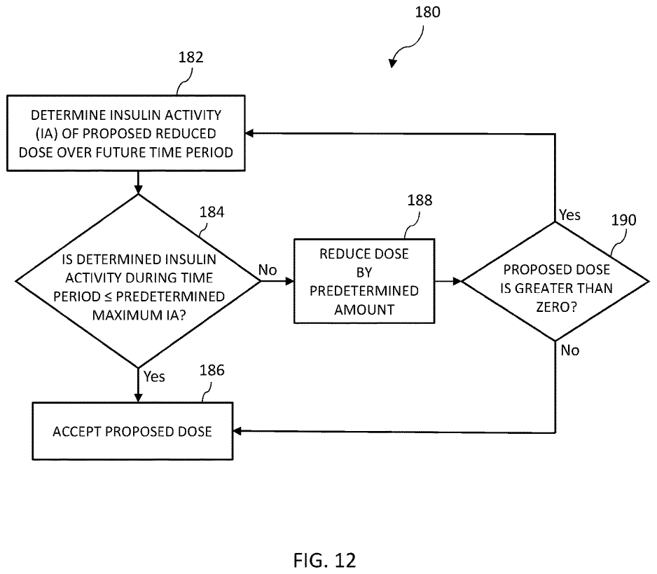

FIG. 12 is a diagrammatic illustration of a method 180 that may be used to verify and/or adjust the proposed reduced dose in connection with step 80 in FIG. 5. In the embodiments shown in FIG. 12, the verification is an iterative process that uses the insulin activity module 18 (as shown in FIG. 1) to determine and predict the insulin activity in the subject that will result from or be produced by the proposed dose over a predetermined period of time in the future (e.g., following the administration of the proposed dose). The proposed dose is verified, and accepted, if the predicted insulin activity levels during the predetermined period of time are within predetermined levels, such as for example a predetermined range of a predetermined maximum insulin activity level. If the method 180 determines that a predicted insulin activity level produced by the proposed dose during the predetermined period of time is greater than the predetermined level, the proposed dose is adjusted by reducing the dose amount. The iterative process is then repeated to verify the adjusted proposed dose. The iterative process repeats until the adjusted proposed dose is verified.

As shown by FIG. 12, at step 182 method 180 determines the insulin activity level of the proposed reduced dose over a future time period. In embodiments, at step 182 the method 180 determines the insulin activity level at each of one or more weekly time periods, such as four weeks into the future. Each of the determined future insulin activity levels is compared to a predetermined maximum insulin activity level at step 184. In embodiments, for example, the predetermined maximum insulin activity level used in step 184 is the target insulin activity level determined at step 168 by method 160 (FIG. 11). If a Yes determination is made at step 184 (e.g., if the predicted insulin activity levels for each of the future four weeks is less than the predetermined maximum, continuing the example above), the proposed reduced dose is verified and accepted as indicated by step 186. If a No determination is made at step 184 (e.g., if any one or more of the four predicted future insulin activity levels is greater than the predetermined maximum), the proposed dose is reduced (e.g., adjusted) at step 188. In embodiments, at step 188 the proposed dose is adjusted by reducing the proposed dose by a predetermined amount. The predetermined amount of the reduction may be based on the amount of the proposed dose. For example, if the proposed dose is greater than or equal to a first amount such as 5.5 mg, the amount of the reduction may be a first reduction amount such as 0.5 mg. If the proposed dose is less than the first amount, the amount of the reduction may be a second and lower amount, such as 0.25 mg.

At step 190 the adjusted proposed dose is checked to determine if it is greater than zero. If a No determination is made at step 190, (e.g., if the proposed dose is zero) the adjusted proposed dose is verified and accepted as the reduced dose 34, as indicated by step 186. For example, a No determination at step 190 means that the recommendation is for no dose, in effect skipping a dose for safety purposes. If a Yes determination is made at step 190, method 180 is repeated, using the reduced proposed dose for the determination at step 182.

### Titration Module

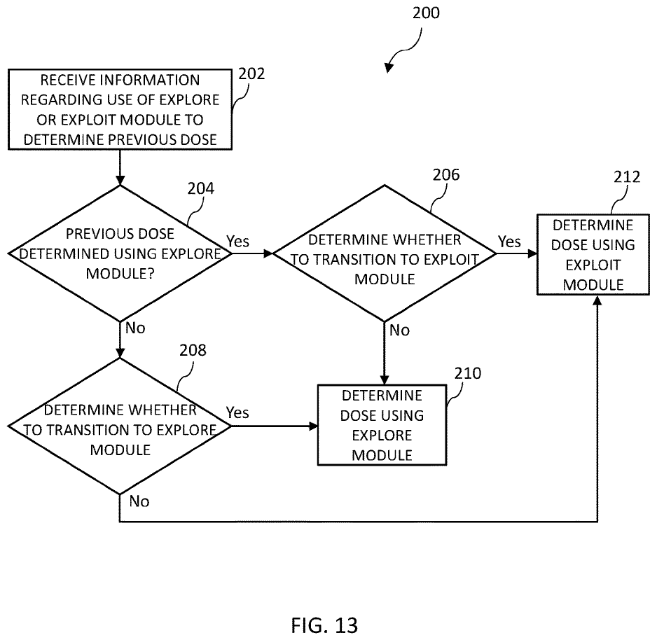

FIG. 13 is a diagrammatic illustration of a method 200 by which the titration module 32 (FIG. 2) of titration dose component 22 may determine whether to use the explore module 37 or the exploit module 38 to determine titration doses 36 of insulin. In embodiments, titration module 22 determines the explore/exploit decision based on factors including one or more of the modules (e.g., which of modules 26, 28, 30, 37 or 38) that was used to determine one or more preceding or earlier doses, and the subject's blood glucose values (e.g., when determining the current dose and values from earlier doses and dose periods).

As shown by steps 202 and 204, in the illustrated embodiments, method 200 makes the explore/exploit decision based on information defining whether the explore module 37 or exploit module 38 was used to determine the first earlier dose. If a Yes determination is made at step 204 (e.g., the first earlier dose was determined by the explore module 37), method 200 determines at step 206 whether to transition to the exploit module 38 to determine the current dose. If a No determination is made at step 204 (e.g., the first earlier dose was determined by the exploit module 38), method 200 determines at step 208 whether to transition to the explore module 37 to determine the current dose. When a No determination is made at step 206 (e.g., to not transition to the exploit module), or Yes determination is made at step 208 (e.g., to transition to the explore module), the method 200 causes the explore module 37 to be used to determine the titration dose 36 as shown by step 210. When a Yes determination is made at step 206 (e.g., to transition to the exploit module), or No determination is made at step 208 (e.g., to not transition to the explore module), the method 200 causes the exploit module 38 to be used to determine the titration dose 36 as shown by step 212.

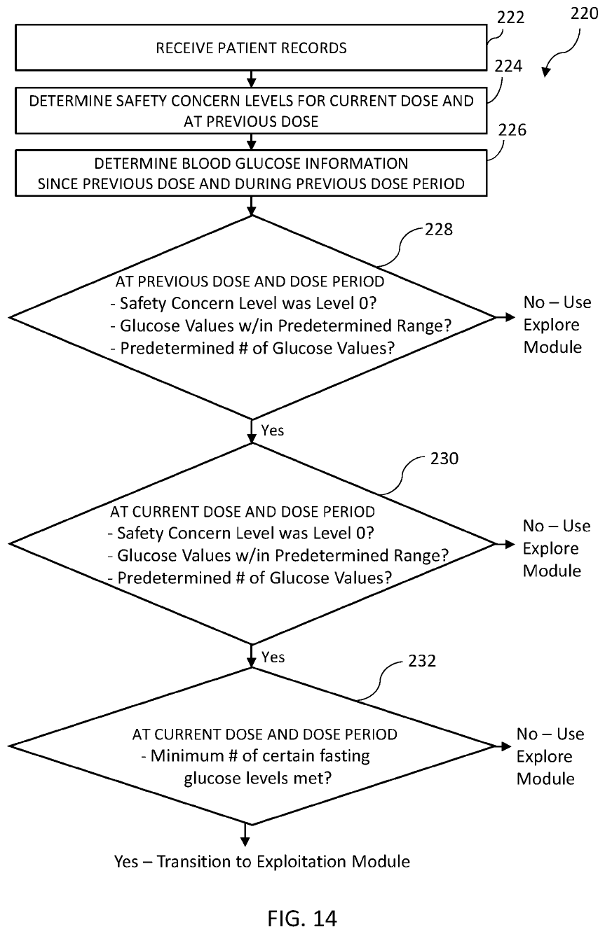

FIG. 14 is a diagrammatic illustration of embodiments of a method 220 by which the titration module 32 may determine whether to switch or transition from use of the explore module 37 to use of the exploit module 38 to determine the titration dose 36 in connection with step 206 (FIG. 13). In the illustrated embodiments, method 220 makes the transition decision based on factors including one or more of the safety concern levels (e.g., determined by method 90) and/or the subject's blood glucose values. Other factors, and/or other methods or approaches, are used in other embodiments.

As shown by step 222, information used by the method 220 may be determined or received from the patient's records 16. The safety concern levels for both the current dose and at least the first earlier dose are determined at step 224. Certain blood glucose measurement information of the subject is determined at step 226. In embodiments, for example, the blood glucose information determined at step 226 may include a determination of the number of fasting blood glucose values are available over a predetermined period of time such as one or more dose periods before the current dose, and a determination of values, such as the median values, of the fasting blood glucose values during the predetermined period of time. In short, assuming that there have been no safety events (e.g., hypo events) and no significant upward or downward trends, the method 220 will increase a dosage amount if a subject's glucose level is above an upper bound of a titration target range and reduce a dosage amount when a subject's glucose level is below a lower bound of the titration target range. Further, the method 220 considers a control target value (e.g., a preferred, specific glucose level), which is used to determine dosage amounts that direct the subject towards the control target value.

At step 228, method 220 determines a first set of certain information relating to the subject at the second earlier dose and during the second earlier dose period. In the illustrated embodiments, method 220 evaluates as criteria at step 228: (1) whether the safety concern level at the time of the second earlier dose was at Level 0, (2) whether there were at least a predetermined number, such as for example three, fasting blood glucose records during the second earlier dose period, and (3) whether the median value of the fasting blood glucose records during the second earlier dose period are within a predetermined range such as 80 mg/dL-120 mg/dL. If a No determination is made that any of the evaluated criteria is not met at step 228, method 220 causes the titration module 32 to not transition, and to use the explore module 32 to determine the titration dose 36 (e.g., as shown by step 210 in FIG. 13).

If at step 228 the method 220 makes a. Yes determination that the first set of criteria are met, the method determines at step 230 a second set of certain information relating to the subject at the first earlier dose and during the first earlier dose period. In the illustrated embodiments, method 220 evaluates as criteria at step 230: (1) whether the safety concern level at the time of the first earlier dose was at Level 0, (2) whether there were at least a predetermined number, such as for example three, fasting blood glucose records during the first earlier dose period, and (3) whether the median value of the fasting blood glucose records during the first earlier dose period is within a predetermined range such as 80 mg/dL-120 mg/dL. If a No determination is made that any of the evaluated criteria is not met at step 230, method 220 causes the titration module 32 to not transition, and to use the explore module 32 to determine the titration dose 36 (e.g., as shown by step 210 in FIG. 13).

If at step 230 the method 200 makes a Yes determination that the second set of criteria are met, the method determines at step 232 a third set of certain information relating to the subject at the first earlier dose and during the first earlier dose period. In the illustrated embodiments, method 220 evaluates as criteria at step 232 whether there have been a certain number (e.g., two in an example embodiment) of minimum fasting blood glucose records in the past two dose periods that are less than a control target value (e.g., 90 or 95 mg/dl in an example embodiment). If a No determination is made at step 232, method 220 causes the titration module 32 to not transition, and to use the explore module 32 to determine the titration dose 36.

If at step 232 the method 220 makes a Yes determination that the second set of criteria are met, the method causes the titration module 32 to transition and to use the exploit module 38 to determine the titration dose 36 (e.g., as shown by step 210 in FIG. 13). By these embodiments, the criteria to switch from the exploration module to the exploitation module depends on consecutive observations of two dosing periods. If the FBGs stay in range and on target for two dosing periods without safety concerns, the method considers the current IA level optimal and determines to maintain the level. For example, if a subject's glucose level is at 110 mg/dL with minimum FBG readings of 86 and 83 mg/dL, the method 220 is designed to recognize that there is insufficient room left to titrate further and so a transition from the explore module 37 to the exploit module 38. However, if a subject's glucose level is at 110 mg/dl and the minimum FBGs readings are 100 and 96 mg/dL, the explore module 37 will continue to be used to determine dosage amounts and increase the dosage amount.

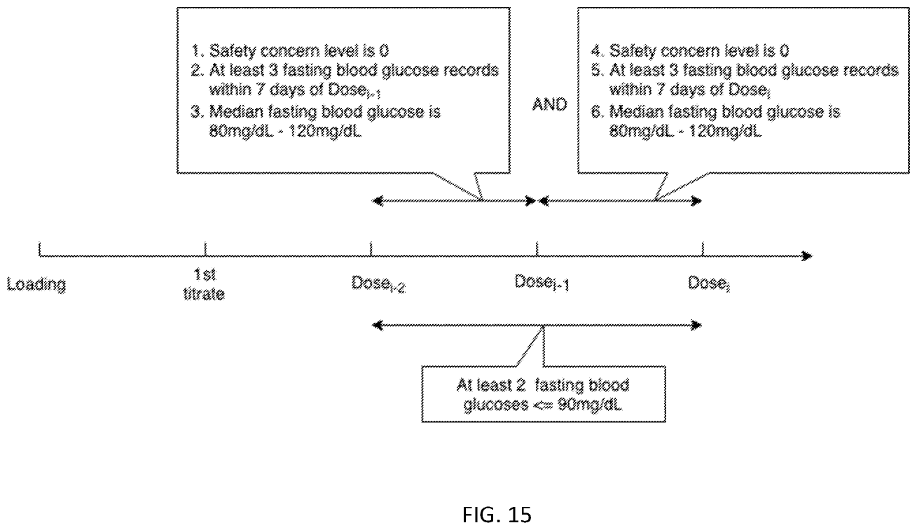

FIG. 15 is a diagrammatic illustration of the criteria such as that described above in connection with method 200 that can be used to determine whether to transition from use of the explore module 37 to use of the exploit module 38 in accordance with embodiments of step 206 (FIG. 13).

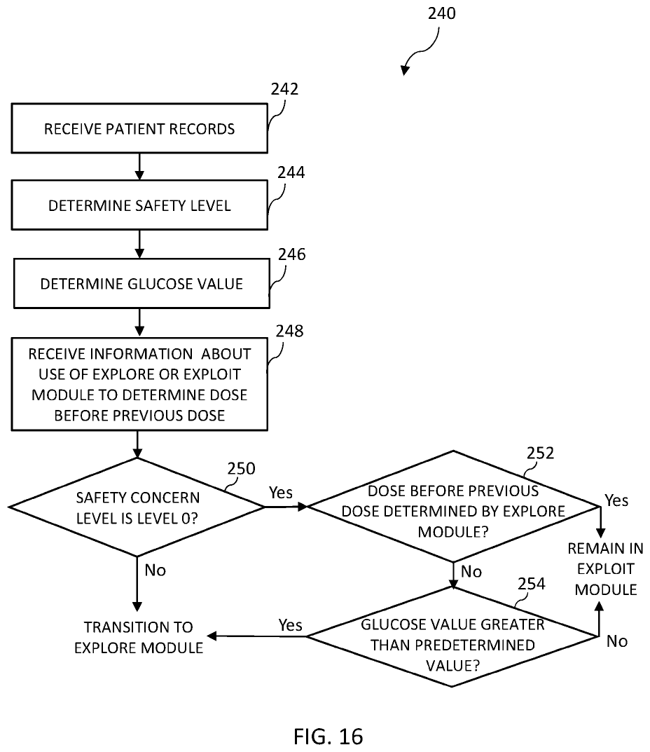

FIG. 16 is a diagrammatic illustration of embodiments of a method 240 by which the titration module 32 can determine whether to transition to use of the explore module 32 to determine the titration dose 36 if the previous dose was determined using the exploit module 38 in connection with step 208 (FIG. 13). In embodiments, method 240 makes the transition decision based on factors including one or more of one or more safety concern levels and/or the subject's blood glucose values. Other factors, and/or other methods or approaches, are used in other embodiments.

As shown by step 242, information used by the method 240 may be determined or received from the patient's records 16 (as shown in FIG. 1). The safety concern level for the current dose is determined at step 244 (e.g., by method 90), and may have been previously determined in connection with the operation of the safety module 30 as described above. Certain blood glucose measurement information of the subject is determined at step 246. Method 240 is also based on whether the second earlier dose was determined using the explore module 37 or the exploit module 38 and receives at step 248 information relating to the nature of that earlier use.

At step 250, method 240 determines the safety concern level. In these illustrated embodiments, method 240 determines whether the safety level concern is Level 0 at step 250. If a No determination is made at step 250, method 240 causes titration module 32 to transition back to the use of the explore module 37 to determine the titration dose 36.

If a Yes determination is made at step 250, method 240 determines at step 252 whether the second previous dose was determined by the explore module 32. If a No determination is made at step 252, method 240 causes the titration module 32 to remain in or continue with the use of the exploit module 38 to determine the titration dose 36.

If a No determination is made at step 252, method 240 determines glucose values at step 254. In the illustrated embodiments, at step 254 method 240 determines whether certain glucose values, such as for example the median fasting blood glucose values during the first previous dosing period and the second previous dosing period, are greater than a predetermined value (e.g., 110 mg/dL) indicating a relatively high glucose level. If a Yes determination is made at step 254, method 240 causes the titration module 32 to remain in or continue with the use of the exploit module 38 to determine the titration dose 36. Is a Yes determination is made at step 254, method 240 causes titration module 32 to transition back to the use of the explore module 37 to determine the titration dose 36.

Criteria used by the titration module 32 to make the explore module 37 or exploit module 38 determination in accordance with some embodiments can be summarized as follows. The titration module 32 may use the explore module 37 to determine the titration dose 36 under certain conditions such as (1) there was no previous use of the exploit module 38 to determine the titration dose, or (2) the safety concern level is less than a predetermined level such as Level 2 representing a relatively low level of concern, or (3) blood glucose values are outside of a predetermine range, such as for example 80 mg/dL-110 mg/dL, representative of a normal range. In these and other embodiments, the titration module 32 may use the exploit module 38 to determine the titration dose 36 under certain conditions such as if the previous titration dose was determined using the explore module 37, if (1) the safety concern level is a predetermined level such as Level 0 representing a relatively low level of concern, and (2) a predetermined number of glucose measurements are available over a predetermined period of time such as three measurements during each of the two preceding dosing periods, and (3) the values of the blood glucose measurements, such as for example the median fasting blood glucose values, are within a predetermined range, such as for example 80 mg/dL-110 mg/dL, representative of a normal range. In these and other embodiments, the titration module 32 may use the exploit module 38 to determine the titration dose 36 under certain conditions such as if the previous dose was determined using the exploit module 38, if the safety concern level is a predetermined level such as Level 0 representing a relatively low level of concern. In these and other embodiments, the titration module 32 may use the exploit module 38 to determine the titration dose 36 under certain conditions such as if the previous two doses were determined using the exploit module 38, if (1) the safety concern level is a predetermined level such as Level 0 representing a relatively low level of concern, and (2) the values of the blood glucose measurements, such as for example the median fasting blood glucose values, are within a predetermined range, such as for example 80 mg/dL-110 mg/dL, over a predetermined period of time, such as for example two dosing periods

### Explore Module

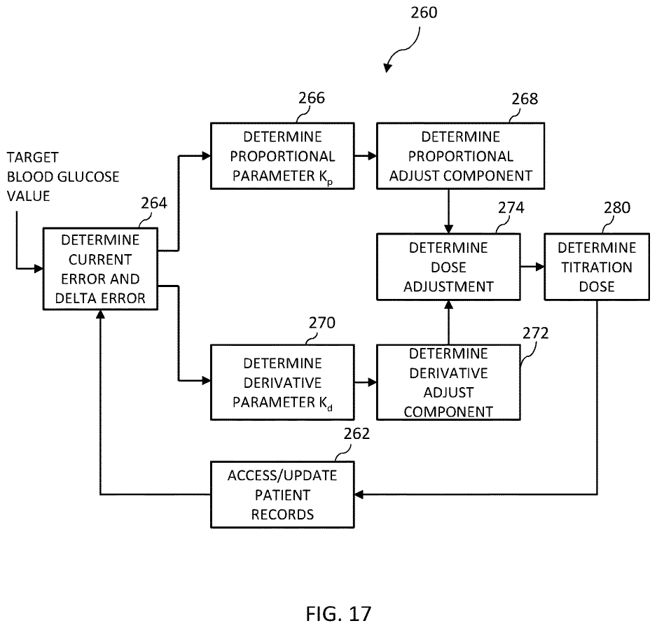

FIG. 17 is a diagrammatic illustration of a method 260 that can be performed by the explore module 37 (FIG. 2) to determine titration doses 36 in accordance with embodiments. In the illustrated embodiments method 260 implements a closed loop control approach based on factors or criteria including a setpoint or target blood glucose value, the first earlier recommended titration dose, and one or more error values defining differences between the first earlier and other previously recommended titration doses (e.g., the second earlier dose). A proportional-derivative control approach is used in the embodiments described in connection with FIG. 17. Other embodiments use other control approaches and/or other criteria.

As shown by FIG. 17, method 260 uses information received from and updates the patient records 16 as represented by step 262. Information received by accessing the patient records 16 at step 262 may include, for example, patient glucose measurements and previously recommended doses. At step 264, method 260 determines error values Current Error and Delta Error based on a setpoint or target blood glucose value for the patent, such as for example ninety mg/dL, and measured blood glucose values such as those received by step 262. At step 266, method 260 determines a first or proportional control parameter Pk. At step 268, a second or proportional adjust component is determined based on the proportional control parameter Pk and the Current Error. At step 270, method 260 determines a second or derivative control parameter Pd. At step 272, a second or derivative adjust component is determined based on the derivative control parameter Pd and the Delta Error. A dose adjustment amount is determined at step 274 based on the proportional adjust component and the derivative adjust component. In embodiments, for example, the dose adjust component is determined at step 274 based on a sum of the proportional adjust component and the derivative adjust component. The proposed recommended titration dose 36 is determined at step 280 based on the dose adjustment amount determined at step 274 and the first earlier dose amount. The first earlier dose amount may be determined at step 262 (e.g., received from the patients records 16 (as shown in FIG. 1)). In embodiments, the recommended titration dose determined at step 280 based on a sum of the dose adjustment amount and the first earlier dose amount. As indicated by step 262, the titration dose 36 determined at step 280 may be stored in the patient records 16. Information in the patient records 16 such as the previous doses amounts and blood glucose measurements during the previous dosing periods is retrieved and used by method 260 as feedback information to enable the closed-loop approach of the method.

At step 264, as noted above, method 260 calculates the Current Error and Delta Error values. In embodiments, the Current Error values are determined based on information including the number and values of blood glucose measurements during preceding dose periods (e.g., everything up to the current dose calculation in embodiments), the target blood glucose value, the number of titration doses that were previously determined, and a regression formula. Table 6 below defines methods and equations that can be used for Current Error determinations at step 264 in embodiments.

TABLE 6
Current Error Determination

| Dose No. | Equation |
|---|---|
| First | (Median FBG value following loading dose) − |
| | (Target Value) |
| Second | (Median FBG value following first titration dose) − |
| | (Target Value) |

| | No. of FBG Values | |
|---|---|---|
| | Following | |
| | Preceding Dose | Equation |
| Third and | 3 or more | Regression Formula |
| Subsequent | 1 or 2 | (Median FBG value following |
| | | preceding titration dose) − |
| | | (Target Value) |

For example, for the first titration dose 36, the Current Error can be determined by subtracting the target glucose value from a median fasting blood glucose value where the median value is determined from blood glucose values measured after the loading dose, and within a predetermined time period, such as seven days, of the first titration dose. For the second titration dose 36, the Current Error can be determined by subtracting the target glucose value from a median fasting blood glucose value where the median value is determined from blood glucose values measured after the first titration dose, and within a predetermined time period, such as seven days, of the second titration dose. Third and subsequent titration doses can be determined based on the number of fasting blood glucose measurements after the previous titration dose, and within a predetermined time period, such as seven days, of the titration dose being determined. In embodiments, for example, if the number of measurements is greater than or equal to a predetermined number such as three, a regression approach may be used to determine the Current Error. If the number of measurements is less than the predetermined number, such as for example one or two measurements continuing with the example above, the Current Error can be determined by subtracting the target glucose value from the median of those fasting blood glucose values.

Regression formulas that can be used to determine the Current Error in the manner described above may follow a structural risk minimization framework, such as for example a framework that seeks a function that minimizes Eq. 4 below:

$${min}_{f} H \vert f \vert {{\sum }_{i = 1}}^{L} {‖ {y}_{i} - f ( {x}_{i} ) ‖}^{2} + λ {‖ Pf ‖}^{2} \qquad Eq . 4$$

In the context of the titration module 32, yᵢ is the value of fasting blood glucose, xᵢ is the number of days passed since the loading dose when yᵢ is created, and L is the number of fasting blood glucose records. The term ∥yᵢ−ƒ(xᵢ)∥² measures the closeness to the data and the regularization operator P controls the model complexity.

Approaches such as that represented by Eq. 4 effectively estimate trends in the blood glucose values, and filter or smooth out outlier values. Other embodiments for calculating Current Error values in connection with step 264 may use other approaches.

At step 264, method 260 determines a value that may be referred to as Previous Error for purposes determining the Delta Error. In embodiments, the Previous Error values are determined based on information including the number and values of blood glucose measurements during preceding dose periods, the target blood glucose value, the number of titration doses that were previously determined, and a regression formula. Table 7 below defines methods and equations that can be used for Previous Error determinations at step 264 in embodiments.

TABLE 7
Previous Error Determination

| Dose No. | Equation |
|---|---|
| First | Current Error |
| Second | (Median FBG value following loading dose) − |
| | (Target Value) |
| Third | (Median FBG value following first titration dose) − |
| | (Target Value) |

| | No. of FBG Values | |
|---|---|---|
| | Following Two | |
| | Preceding Doses | Equation |
| Fourth and | 3 or more | Regression Formula |
| Subsequent | 1 or 2 | (Median FBG value following |
| | | preceding titration dose) − |
| | | (Target Value) |

For example, for the first titration dose 36, the Previous Error can be defined as the Current Error described above. For the second titration dose, the Previous Error can be determined by subtracting the target glucose value from a median fasting blood glucose value where the median value is determined from blood glucose values measured after the loading dose, and within a predetermined time period, such as seven days, of the first titration dose. For the third titration dose 36, the Previous Error can be determined by subtracting the target glucose value from a median fasting blood glucose value where the median value is determined from blood glucose values measured after the first titration dose, and within a predetermined time period, such as seven days, of the second titration dose. Fourth and subsequent titration doses can be determined based on the number of fasting blood glucose measurements after the previous titration dose, and within a predetermined time period, such as seven days, of the titration dose being determined. In embodiments, for example, if the number of measurements is greater than or equal to a predetermined number such as three, a regression approach such as that described above in connection with the Current Error (e.g., using Eq. 4) may be used to determine the Previous Error. If the number of measurements is less than the predetermined number, such as for example one or two measurements continuing with the example above, the Previous Error can be determined by subtracting the target glucose value from the median of those fasting blood glucose values.

The Delta Error is determined based on the Current Error and the Previous Error, such as for example by Eq. 5 below:

$$Delta Error = Current Error - Previous Error \qquad Eq . 5$$

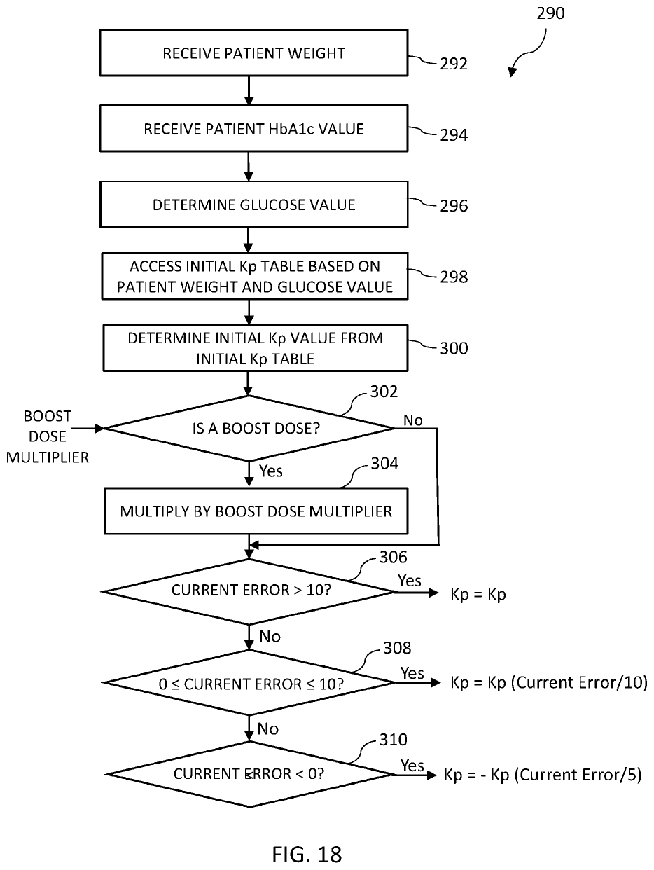

FIG. 18 is a diagrammatic illustration of a method 290 that can be used by the explore module 37 to determine the proportional parameter Pk in connection with step 266 of method 260 (FIG. 17). Proportional parameter Pk functions as a gain parameter to reduce differences between current and target blood glucose levels in the control method 260. Proportional parameter Pk is adaptive based on parameters such as information characteristic of the subject and information characteristic of the subject's dosing history and physiological responses to the dosing history. As shown by steps 292 and 294, respectively, the illustrated embodiments of method 290 receive and use the subject's weight and measured HbA1c values (e.g., from patient records 16 (as shown in FIG. 1)) as a basis for determining the proportional parameter Pk. At step 296 the method 290 determines a blood glucose value for the subject. In embodiments, method 290 determines at step 296 a baseline fasting blood glucose value (BFBG) based on the subject's HbA1c value determined at step 294. The method may be based upon historical basal insulin trials using regression methods, for example. Eq. 6 below is an example of an equation by which the baseline fasting blood glucose level can be determined based on the HbA1c level and predetermined numerical values.

$$Baseline Fasting Blood Glucose Level = ( HbA 1 c - 4.848 ) \times 18 / 0.373 \qquad Eq . 6$$

As shown by step 298, method 290 makes use of a stored initial Kp table that defines a range of values for Kp based on factors such as the subject's weight and the baseline fasting blood glucose level (BFBG) determined at step 296 (e.g., a lookup table). Table 8 below is an example of an initial Kp value table in accordance with embodiments. As shown, the initial values of Kp are indexed based on ranges of BFBG values and ranges of subject weights.

TABLE 8
Initial Kp Value Table

| | FBG < 160 | 160 mg/dL ≤ FBG < | BFBG ≥ 200 |
|---|---|---|---|
| | mg/dL | 200 mg/dL | mg/dL |
| Weight < 80 kg | 0.08 | 0.10 | 0.12 |
| Weight ≥ 80 kg | 0.10 | 0.12 | 0.14 |

At step 300, the initial Kp value is determined by accessing the initial Kp value table. Other embodiments of method 290 determine the initial Kp value using other approaches and/or parameters and values.

As shown by step 302, method 290 determines whether the dose determined by the explore module 37 should be boosted (e.g., a boost dose). In embodiments, method 290 determines that the dose is a boost dose if a boost multiplier is used. If a Yes determination is made at step 302, method 290 increases the initial Kp value by a predetermined amount. In the illustrated embodiments, method 290 multiplies the initial Kp value by the boost multiplier as shown by step 304. If a No determination is made at step 302, the initial Kp value is not adjusted by step 304.

As shown by steps 306, 308 and 310, the initial Kp value may also be adjusted based on a factor such as the Current Error value. At step 306 method 290 determines whether the Current Error is within a first range, such as for example greater than a first value such as ten. If a Yes determination is made at step 306, the initial Kp value (with any boost adjustment) is determined as the Kp value in connection with step 266 of method 260 (FIG. 17). Eq. 7.1 below defines the determination of the Kp value in connection with a Yes determination at step 306 in embodiments.

$$Kp = Kp ( initial , with any boost adjustment ) \\ When : Current Error > 10 \qquad Eq . 7.1$$

If a No determination is made at step 306, method 290 determines at step 308 whether the Current Error is within a second range less than the first range, such as for example greater than zero and less than or equal to ten when continuing the example above. If a Yes determination is made at step 308, the initial Kp value (with any boost adjustment) is determined based on that initial Kp value and a first factor including the Current Error as the Kp value in connection with step 266 of method 260. Eq. 7.2 below defines the determination of the Kp value in connection with a Yes determination at step 308 in embodiments.

$$Kp = Kp ( initial , with any boost adjustment ) \times Current Error / 10 \\ When : 0 \leq Current Error \leq 10 \qquad Eq . 7.2$$

If a No determination is made at set 308, method 290 determines at step 310 that the Current Error is within a third range less than the second range, such as for example less than zero when continuing the example above. If a Yes determination is made at step 310, the initial Kp value (with any boost adjustment) is determined based on the initial Kp value and a second factor including the Current Error as the Kp value in connection with step 266 of method 260. Eq. 7.3 below defines the determination of the Kp value in connection with a Yes determination at step 310 in embodiments.

$$Kp = Kp ( initial , with any boost adjustment ) \times Current Error / 5 \\ When : Current Error < 0 \qquad Eq . 7.3$$

In other embodiments, the proportional parameter Kp is determined by other approaches and/or using other values.

Referring back to step 268 in FIG. 17, the proportional adjust component used in connection with the method 260 is determined based on factors including the proportional parameter Kp and the Current Error. Eq. 8 below defines the determination of the proportional adjust component in accordance with embodiments.

$$Proportional adjust parameter = Kp \times Current Error \qquad Eq . 8$$

As noted above, in connection with step 270, method 260 determines the derivative parameter Pd used by the control approach used by the explore module 37 to determine titration doses 36. Derivative parameter Pd functions as a slope parameter to enhance linear prediction and to reduce overshooting in the control method 260. Derivative parameter Pd is adaptive based on parameters such as the error values determined at step 264. In embodiments, method 260 determines the derivative parameter Pd based on factors including the values of the Current Error. Eq. 9.1 below defines the determination of the Kd value when the Current Error is within a first predetermined range, such as for example greater than zero, in embodiments.

$$Kd = 0 \\ When : Current Error > 0 \qquad Eq . 9.1$$

Eq. 9.2 below defines the determination of the Kd value when the Current Error is with a second predetermined range, such as a range outside of the first predetermined range, in embodiments.

$$Kd - 0.15 \\ When : Current Error \leq 0 \qquad Eq . 9.2$$

In other embodiments, the derivative parameter Kd is determined by other approaches and/or using other values.

At step 272, the derivative adjust component used in connection with the method 260 is determined based on factors including the derivative parameter Kd and the Delta Error. Eq. 10 below defines the determination of the derivative adjust parameter in accordance with embodiments.

$$Derivative adjust parameter = Kd \times Delta Error \qquad Eq . 10$$

### Boost Dose

As described above in connection with FIG. 18, embodiments of the explore module 37 may determine to recommend a boost dose under certain circumstances (e.g., by increasing the proportional parameter Kp in method 260, which leads to a relatively larger dose adjustment with respect to the previous dose). The boost dose provides the system the flexibility to increase the dose more rapidly when determined to be necessary. The boost dose facilitates a subject more quickly reaching a target glucose range in some situations. For example, when the subject misses a weekly dose, the explore module 37 may determine in some cases that a boost dose is necessary to reach the target glucose range more quickly. Factors that may be used by method 260 to determine whether to recommend a boost dose and/or the amount of the boost dose (e.g., the boost dose multiplier in FIG. 18) include one or more of the safety concern level, and the number and amounts of available blood glucose measurements (e.g., in the patient records 16 of FIG. 1) within a predetermined period of time of the previous dose determination.

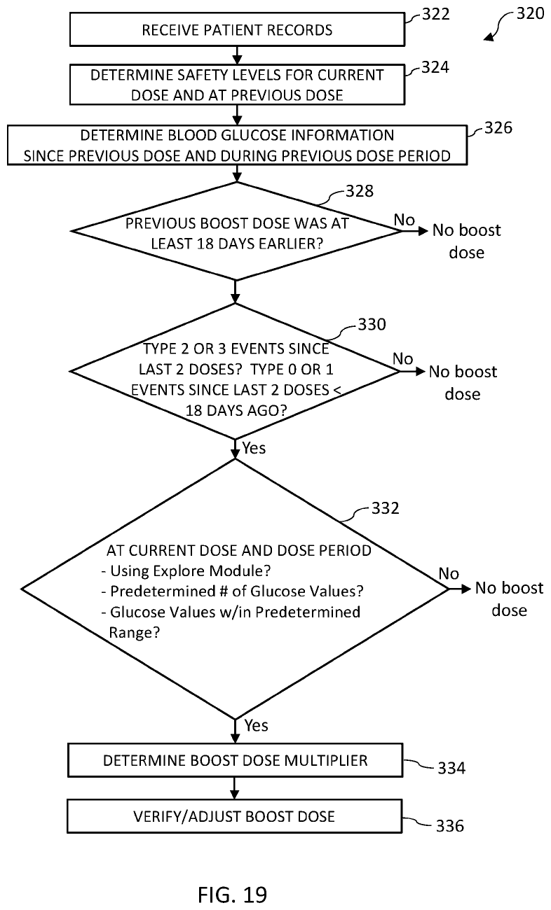

FIG. 19 is a diagrammatic illustration of embodiments of a method 320 by which the explore module 37 may determine whether to recommend a boost dose, and the amount of any such boost dose. Method 320 makes the boost dose decision based on factors including one or more safety concern levels (e.g., determined by method 90) and/or the subject's blood glucose values. As shown by step 322, information used by the method 320 may be determined or received from the patient records 16 (as shown in FIG. 1). The safety concern levels for both the current dose and at least the first earlier dose are determined at step 324. Certain blood glucose measurement information of the subject is determined at step 326. In embodiments, for example, the blood glucose information determined at step 326 may include a determination of the number of fasting blood glucose values available over a predetermined period of time such as one or more dose periods before the current dose, and a determination of values, such as the median values, of the fasting blood glucose values during the predetermined period of time.

At step 328, method 320 determines a first set of information relating to the subject during a predetermined number of days previous. In the illustrated embodiments, method 320 evaluates at step 328 whether a most recently recommended boost dose (e.g., the last boost dose) was at least 18 days before the determination of the current dose. If a No determination is made at step 328, method 320 determines that no boost dose is recommended.

If at step 328 the method 320 makes a Yes determination that the first set of criteria is met, the method determines at step 330 a second set of certain information relating to the subject from the past two dosing periods. In the illustrated embodiments, method 320 evaluates as criteria at step 330 whether (1) any type 2 or type 3 events have occurred since the last two doses or (2) any type 0 or type 1 events have occurred since the last two doses but no more than 18 days ago. If a No determination is made at step 330, method 320 determines that no boost dose is recommended.

If at step 330 the method 320 makes a Yes determination that the second set of criteria is met, the method determines at step 332 a third set of certain information relating to the subject during the first earlier dose period. In the illustrated embodiments, method 320 evaluates as criteria at step 332 (1) whether the titration module 32 is using the explore module 37, (2) whether there are at least a predetermined number, such as for example three, fasting blood glucose records during the first earlier dose period, (3) whether the median value of the fasting blood glucose records during the first earlier dose period are within a predetermined range such as greater than or equal to 130 mg/dL, and (4) whether the minimum value of the fasting blood glucose records during the first earlier dose period are within a second predetermined range such as greater than or equal to 85 mg/dL. If a No determination is made that any of the evaluated criteria is not met at step 332, method 320 determines that no boost dose is recommended.

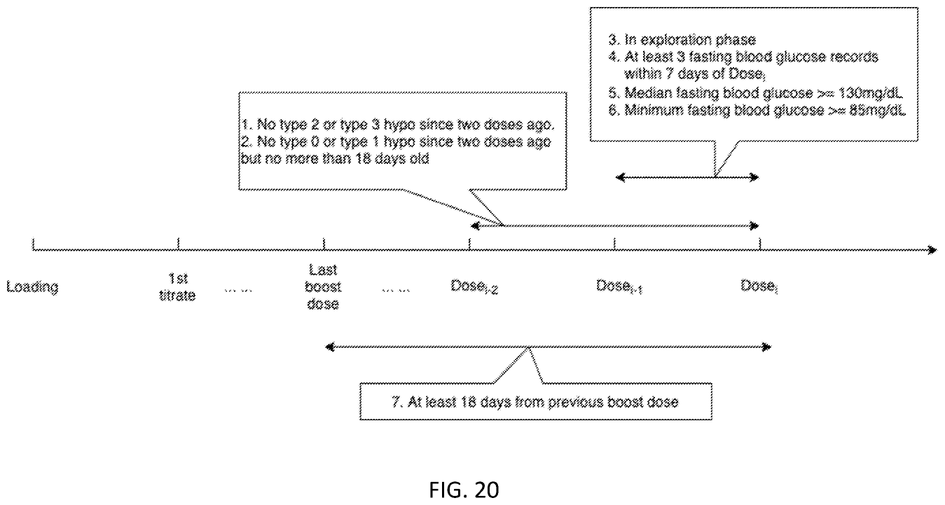

If at step 332 the method 320 makes a Yes determination that the third set of criteria are met, the method causes the explore module 37 to recommend a boost dose. FIG. 20 is a diagrammatic illustration of a summary of the criteria described above in connection with method 320 that can be used to determine whether to recommend a boost dose.

Referring back to FIG. 19, if at step 332 the method 320 makes a Yes determination causing the explore module 37 to recommend a boost dose, at step 334 the method determines the amount of the boost dose. In embodiments, method 320 determines the amount of the boost dose at step 334 based on factors such as the number of blood glucose values during the first previous dose period in the patient records 16 (FIG. 1) within a predetermined period of time such as the first earlier dose period. Table 9 below (e.g., a lookup table) defines exemplary amounts of boost determined by the method 320 at step 334 in embodiments (e.g., in terms of exemplary multipliers of the initial proportional parameter Pk determined at step 266 of method 260 (FIG. 17)).

TABLE 9
Boost Multiplier

| No. of Blood Glucose Values | Multiplier |
|---|---|
| 3, 4 | 2 |
| ≥5 | 3 |

In embodiments of explore module 37, titration doses 36 determined using a boost multiplier (e.g., at step 302 of method 290 (FIG. 18)) are verified and/or adjusted before being recommended to the subject as indicated by step 336. In embodiments, a verification and adjustment method similar to method 180 described above in connection with FIG. 12 can be used to verify and adjust boost doses before they are recommended to the subject in connection with step 336. In connection with such a verification and adjustment process used for boost doses, the future time period over which the insulin activity of the proposed dose is predicted or determined at step 182 shown in FIG. 12 may be one dose period such as one week into the future. The determined maximum insulin activity (IA) level that is used at step 184 in FIG. 12 may be determined based on the blood glucose levels of the subject. In embodiments, for example, the maximum insulin activity level used at step 184 in FIG. 12 is determined based on ranges of the median FBG of the subject during the first earlier dose period. Table 10 below defines maximum insulin activity levels that can be used at step 184 in FIG. 12 of method 180 in embodiments for purposes of verifying and adjusting boost doses.

TABLE 10
Maximum IA Levels for Boost Dose Verification and Adjustment

| Median Fasting Blood Glucose Values | Maximum IA |
|---|---|
| FBG ≥ 130 mg/dL | 1.3 × maximum IA during |
| | first earlier dose period |
| FBG ≤ 110 mg/dL | 1.2 × maximum IA during |
| | first earlier dose period |
| 110 mg/dL > FBG < 130 mg/dL | Median FBG × 0.005 + 0.65 |

### Exploit Module

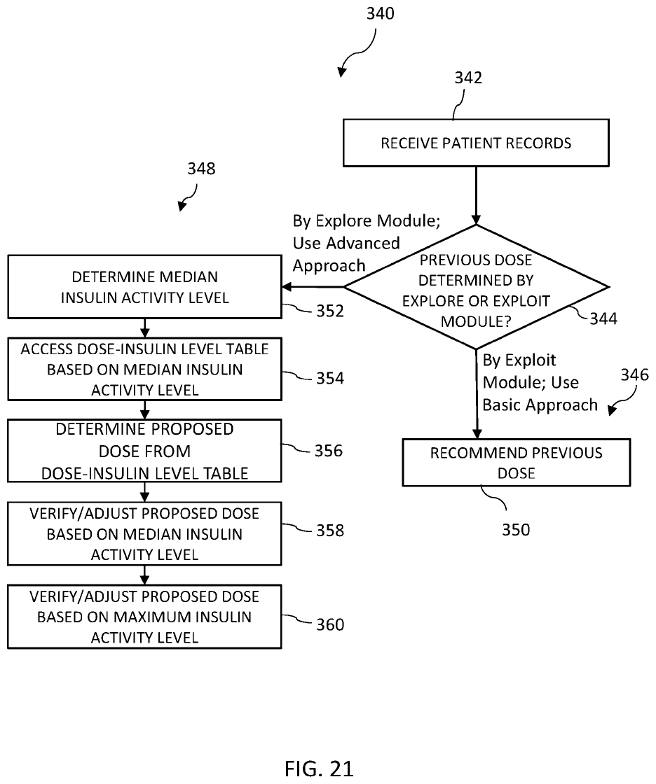

FIG. 21 is a diagrammatic illustration of a method 340 that can be used by the exploit module 38 to determine proposed recommended titration doses 36 in embodiments. As shown, method 340 accesses patient records at step 342 and determines at step 344 whether to use a basic or first approach 346 or an advanced or second approach 348 based on information in the patient records. In the illustrated embodiments, the determination at step 344 is based on information representative of whether the first earlier dose was determined using the explore module 37 or the exploit module 38. Other embodiments use other criteria for making the determination at step 344.

If an exploit module determination is made at step 346 (e.g., the first earlier dose was determined using the exploit module), method 340 recommends the previous or first earlier dose as the titration dose 36 as indicated by step 350 (e.g., to continue with the previous dose).

If an explore module determination is made at step 344, method 340 determines the titration dose 36 by the exploit module 38 using the second approach 348. In embodiments of the second approach 348, method 340 determines an initial proposed dose based on the subject's glucose measurement values, and then verifies and adjusts the initial proposed dose by a first round process based on predicted future values of the median insulin activity levels produced by the doses, and by a second round process based on predicted future values of the maximum insulin activity levels produced by the doses.

As shown at step 352, the second approach 348 determines the subject's median insulin activity level over a predetermined period of time, such as for example during the first earlier dosing period and the second earlier dosing period. The predetermined period of time may, for example, be within a predetermined maximum period of time, such as fourteen days, of the determination of the initial proposed dose. The median insulin activity level can, for example, be determined by the Insulin Activity Module 18 using information in the patient records 16 (as shown in FIG. 1), such as the dosing history over the predetermined period of time. As shown by step 354, method 340 accesses a dose—insulin activity level table, such as Table 2 described above in connection with the Insulin Activity Module 18, based on the median insulin activity level. An initial proposed insulin dose is determined from the dose—insulin activity table at step 356.

At step 358 the initial proposed dose is verified and/or adjusted based on a first parameter or factor, such as predicted median insulin activity levels produced by the initial proposed dose. In embodiments, an iterative verification and adjustment process is performed at step 358 until certain predetermined acceptance criteria are met. In the illustrated embodiments the verification and adjustment process at step 358 predicts median insulin activity levels for a predetermined period of time, such as for example four weeks, based on a hypothetical administration of the proposed dose during dosing periods (e.g., weekly), and checks the predicted median activity levels of each week against the subject's median insulin activity level determined at step 352. During the verification and adjustment process at step 358, the proposed dose amount is increased (e.g., adjusted) if the predicted median insulin activity levels cannot be maintained at the level of the subject's median insulin activity level. The adjustment may be based on the median fasting blood glucose of the first earlier dose period as follows: (1) If median FBG<80 mg/dL, medianIA=medianIA*0.8; (2) If 80 mg/dL<=median FBG<=90 mg/dL, medianIA=medianIA*0.85; (3) If median FBG>90 mg/dL, medianIA=medianIA*0.9. By this approach the predicted median activity levels of each week are checked against an adjusted median insulin activity level. In embodiments, step 358 terminates upon the occurrence of one or more factors, such as when the criteria on the median insulin activity level are met, or when the adjusted proposed dose reaches a predetermined maximum dose, such as for example 30 mg.

At step 360, after the proposed dose is verified and/or adjusted based on median insulin activity levels at step 358, the proposed dose is verified and/or adjusted based on a second parameter or factor, such as predicted maximum insulin activity levels produced the proposed dose. In embodiments, an iterative verification and adjustment process is performed at step 360 until certain predetermined acceptance criteria are met. In the illustrated embodiments the verification and adjustment process at step 360 predicts maximum insulin activity levels for a predetermined period of time, such as for example four weeks, based on a hypothetical administration of the proposed dose during dosing periods (e.g., weekly), and checks the predicted maximum activity levels of each week against a predetermined maximum insulin activity level. During the verification and adjustment process at step 360, the proposed dose amount is decreased (e.g., adjusted) if the predicted maximum insulin activity levels is greater than the predetermined maximum insulin activity level. For example, in embodiments the maximum insulin activity is also adjusted based on the median fasting blood glucose of the first earlier dose period as follows: (1) If median FBG<80 mg/dL, max IA=maxIA (no change); (2) If 80 mg/dL<=median FBG<=90 mg/dl, maxIA=maxIA*1.05; (3) If median FBG>90 mg/dL, maxIA=maxIA*1.1. In embodiments, step 360 terminates upon the occurrence of one or more factors, such as when the criteria on the maximum insulin activity level are met, or when the adjusted proposed dose reaches a predetermined minimum dose, such as for example 0 mg. By the approach reflected in the described embodiments, the exploit module verifies the IA twice. The first run may ensure the maintenance IA level (e.g., a four-week prediction) would be in a desired range. The second run may ensure that peak IA does not exceed a certain level to prevent hypoglycemia.

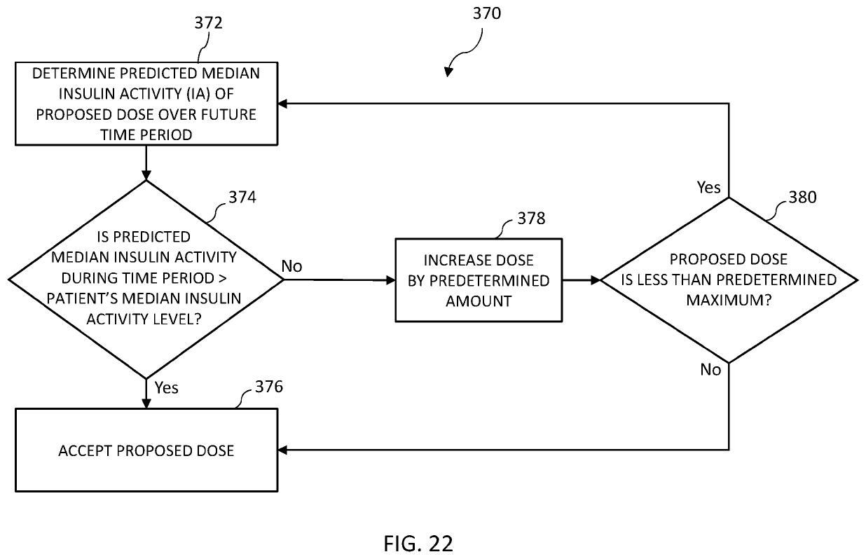

FIG. 22 is a diagrammatic illustration of a method 370 that may be used to verify and/or adjust the proposed initial dose in connection with step 358 of method 340 (FIG. 21). In the embodiments shown in FIG. 22, the verification is an iterative process that uses the insulin activity module 18 to determine and predict the insulin activity in the subject that will result from or be produced by the initial proposed dose over a predetermined period of time in the future (e.g., following the administration of the proposed dose). The proposed dose is verified, and accepted, if the predicted median insulin activity levels during the predetermined period of time are within a predetermined range of the subject's median insulin activity level (e.g., as determined at step 352 of method 340). If the method 370 determines that a predicted median insulin activity level produced by the proposed dose during the predetermined period of time is less than or equal to the subject's median insulin activity level, the proposed dose is adjusted by increasing the dose amount. The iterative process is then repeated to verify the adjusted proposed dose. The iterative process repeats until the adjusted proposed dose is verified.

As shown by FIG. 22, at step 372 method 370 determines the median insulin activity level of the proposed dose over a future time period. In embodiments, at step 372 the method 370 determines the median insulin activity level at each of one or more weekly time periods, such as four weeks into the future. Each of the determined future median insulin activity levels is compared to the subject's median insulin activity level at step 374. If a Yes determination is made at step 374 (e.g., if the predicted median insulin activity levels for each of the future four weeks is greater than the subject's median insulin activity level), the proposed dose is verified and accepted as indicated by step 376. If a No determination is made at step 374 (e.g., if any one or more of the four predicted future median insulin activity levels is less than or equal to the subject's median insulin activity level), the proposed dose is increased (e.g., adjusted) at step 378. In embodiments, at step 378 the proposed dose is adjusted by increasing the proposed dose by a predetermined amount. The predetermined amount of the increase may be based on the amount of the proposed dose. For example, if the proposed dose is less than a first amount such as 5.0 mg, the amount of the increase may be a first increase amount such as 0.25 mg. If the proposed dose is greater than or equal to the first amount, the amount of the increase may be a second and greater amount, such as 0.5 mg.

At step 380 the adjusted proposed dose is checked to determine if it is less than a predetermined maximum dose, such as for example 30 mg. If a No determination is made at step 380, the adjusted proposed dose is verified and accepted as indicated by step 376. If a Yes determination is made at step 380, method 370 is repeated, using the increased proposed dose for the determination at step 372.

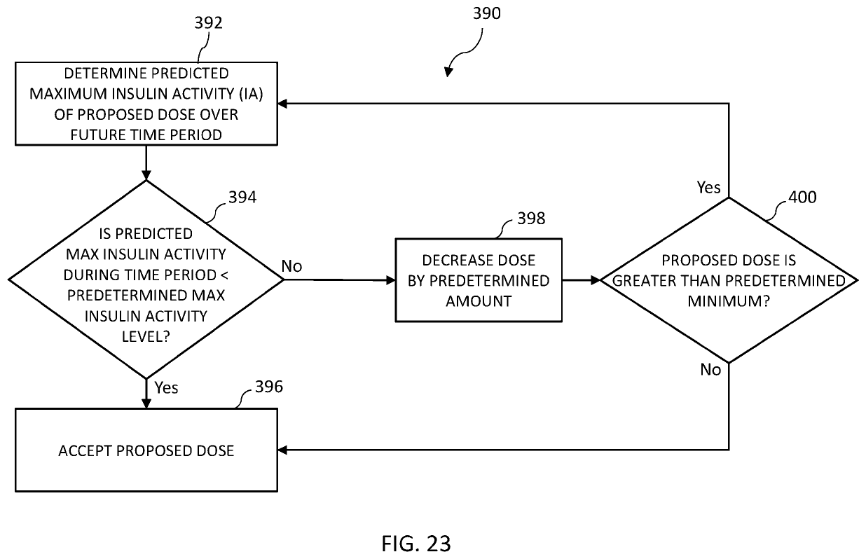

FIG. 23 is a diagrammatic illustration of a method 390 that may be used to verify and/or adjust the proposed reduced dose in connection with step 360 of method 340 (FIG. 21). In the embodiments shown in FIG. 23, the verification is an iterative process that uses the insulin activity module 18 (as shown in FIG. 1) to determine and predict the insulin activity in the subject that will result from or be produced by the proposed dose over a predetermined period of time in the future (e.g., following the administration of the proposed dose). The proposed dose is verified, and accepted, if the predicted insulin activity levels during the predetermined period of time are within a predetermined range of a predetermined maximum insulin activity level. If the method 390 determines that a predicted maximum insulin activity level produced by the proposed dose during the predetermined period of time is greater than or equal to the predetermined maximum insulin activity level, the proposed dose as adjusted by reducing the dose amount. The iterative process is then repeated to verify the adjusted proposed dose. The iterative process repeats until the adjusted proposed dose is verified (e.g., when the adjusted proposed dose becomes about 0 mg).

As shown by FIG. 23, at step 392 method 390 determines the maximum insulin activity levels of the proposed dose over a future time period. In embodiments, at step 392 the method 390 determines the maximum insulin activity level at each of one or more weekly time periods, such as four weeks into the future. Each of the determined future maximum insulin activity levels is compared to the predetermined maximum insulin activity level at step 394. If a Yes determination is made at step 394 (e.g., if the predicted maximum insulin activity levels for each of the future four weeks is less than the predetermined maximum), the proposed dose is verified and accepted as indicated by step 396. If a No determination is made at step 394 (e.g., if any one or more of the four predicted future insulin activity levels is greater than or equal to the predetermined maximum), the proposed dose is reduced or decreased (e.g., adjusted) at step 398. In embodiments, at step 398 the proposed dose is adjusted by reducing the proposed dose by a predetermined amount. The predetermined amount of the reduction may be based on the amount of the proposed dose. For example, if the proposed dose is greater than or equal to a first amount such as 5.5 mg, the amount of the reduction may be a first reduction amount such as 0.5 mg. If the proposed dose is less than the first amount, the amount of the reduction may be a second and lower amount, such as 0.25 mg.

At step 400 the adjusted proposed dose is checked to determine if it is greater than a predetermined minimum value, such as for example zero. If a Yes determination is made at step 400, method 390 is repeated, using the reduced proposed dose for the determination at step 392. If a No determination is made at step 400, (e.g., if the proposed dose is zero) the adjusted proposed dose is verified and accepted as indicated by step 396. In effect, if a No determination is made, the method recommends no dose or skipping a dose. Because a purpose of the method is to switch from the explore module to the exploit module, a precondition of a Level 0 safety concern is already satisfied. The interpretation is based on the previous dosing history, and generally the subject may maintain the current desired insulin activity level without an additional dose. In situations where this is not the case, as time goes by, the insulin activity level in the subject may drop with new additional doses and the method at that time will switch back to the use of the explore module. Although Level 0 is used in embodiments, other values having a lower bound are used in other embodiments.

Referring back to FIG. 21, after the verification and/or adjustment of the dose based upon maximum insulin activity levels at step 360, the proposed dose is recommended as the titration dose 36 (FIG. 2) by method 340. In embodiments, method 340 causes any increases in recommended proposed doses over the first earlier dose to be limited by a predetermined maximum increase, such as for example 2 mg.

Simulation trials have demonstrated that the methods and systems described herein are capable of providing efficacious proposed dosing regimens when used in connection with relatively long-acting insulin.

### System

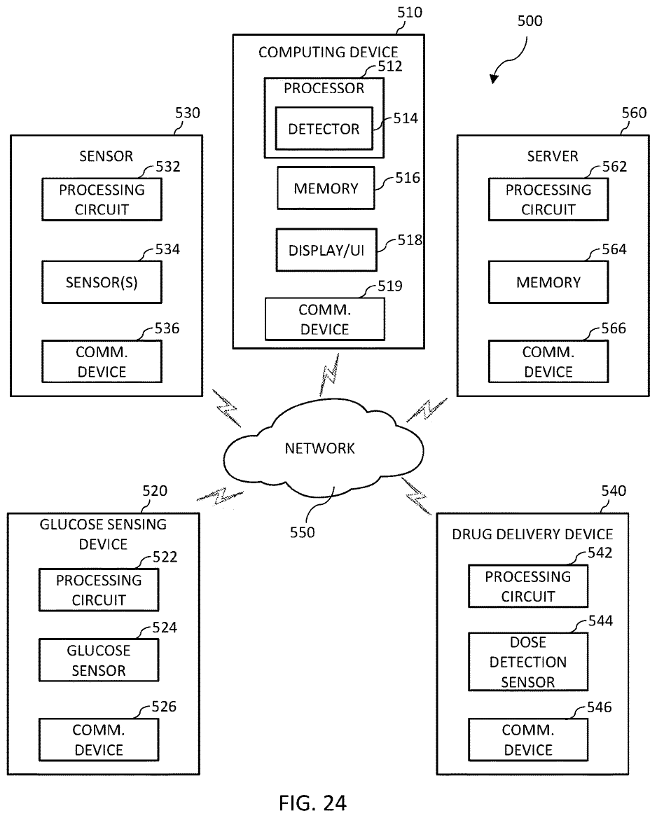

FIG. 24 is a diagrammatic illustration of a system 500 that includes diabetes management system 10 in accordance with embodiments. As shown, system 500 includes one or more computing devices or systems 510 (one is shown for purposes of example), glucose sensing device 520, sensor 530, drug delivery device 540 and server 560 coupled to one another for data communication by network 550. Network 550 is shown as a functional element and may include one or more wired and/or wireless (e.g., RF and/or optical) networks for connecting the computing devices 510, glucose sensing device 520, sensor 530 drug delivery device 540 and server 560. In some embodiments, for example, the network 550 may include one or more local area networks (LAN), the internet, one or more wide area networks (WAN). Local area networks may for example, include WiFi and near field communication networks such as Bluetooth. Wide area networks may for example include cellular networks. Accordingly, although the system components including the computing devices 510, glucose sensing device 520, sensor 530 drug delivery device 540 and server 560 are diagrammatically illustrated in FIG. 24 as being coupled to one another through the network 550, individual components may also be coupled to one another by components of the network. By way of example, computing device 510 may be directly coupled to the glucose sensing device 520 and to the drug delivery device 540 by WiFi and/or Bluetooth components of the network 550.

The diabetes management system 10 of FIG. 1 may be implemented on a mobile app, a cloud, embedded code, other computing device, or some combination. Examples of computing devices 510 include mobile devices, such as a smartphone. Alternatively and additionally, computing devices 510 may include a laptop, desktop, tablet, or server computer, for example. In the illustrated embodiments, computing device 510 includes processor 512, memory 516, display/user-interface (UI) 518, and communication device 519.

Processor 512 includes one or more processors that execute software and/or firmware stored in memory 516 of the computing device 510. The software/firmware code contains instructions that, when executed by the processor 512, causes the processor 512 to perform functions and methods described herein. For example, the functionality of the dosing module 12 and/or the insulin activity module 18 of FIG. 1 may be executed in applications, or apps, of the computing devices 510. At least a portion of the functionality may be implemented on a cloud computing system in communication with the apps (e.g., mobile apps). In some embodiments, the dosing module 12 and insulin activity module 18 may be stored in memory such as memory 516 of one or more computing devices 510 and executed by processor(s) of the one or more computing devices 510 to perform the functions and methods described herein.

Processor 512 may also include detector and/or detector logic 514 operative to implement functionality described herein. Memory 516 may be any suitable computer readable medium that is accessible by processor 512. Memory 516 may be a single storage device or multiple storage devices, may be located internally or externally to processor 512, and may include both volatile and non-volatile media. Exemplary memory 516 includes random-access memory (RAM), read-only memory (ROM), electrically erasable programmable ROM (EEPROM), flash memory, a magnetic storage device, optical disk storage, or other suitable non-transitory or other medium which is configured to store data and which is accessible by processor 512. Memory 516 may include cloud storage.

A display/user interface 518 in communication with processor 512 and operative to provide user input data to the system 500 and to receive and display data, information, and prompts generated by the system. User interface 518 includes at least one input device for receiving user input and providing the user input to the system 500. In embodiments, user interface 518 is a graphical user interface (GUI) including a touchscreen display operative to display data and receive user inputs. The touchscreen display allows the user to interact with presented information, menus, buttons, and other data to receive information from the system 500 and to provide user input into the system. Alternatively and additionally, a keyboard, keypad, microphone, mouse pointer, or other suitable user input device may be provided.

Communication device 519 enables computing device 510 to establish wired and/or wireless communication links with other devices and components of system 500.

Communication device 519 may comprise one or more wireless antennas and/or signal processing circuits for sending and receiving wireless communications, and/or one or more ports for coupling to physical wires for sending and receiving data. Using communication device 519, computing device 510 may establish via the network 550 one or more short-range communication links, including one or more of communication links with glucose sensing device 520 and/or drug delivery device 540.

In embodiments, the memory 516 of the computing device 510 may include instructions for implementing any or all of the methods and functions described above in connection with the dosing module 12. Additionally and alternatively, the memory 516 of the computing device 510 may store information of the types described above that are included in the patient records 16 and/or used by the dosing module 12. The display/UI 518 of the computing device 510 may be used by users such as a physician or other HCP in accordance with any or all of the methods described above in connection with the dosing module 12. For example, a HCP can interface with the computing device 510 through the display/UI 518 to determine insulin doses, such as one or more, or all, of the loading doses, reduced doses and titration doses, for patients. Additionally or alternatively, a patient can interface with the computing device 510 through the display/UI 518 to determine insulin doses, such as one or more, or all, of the loading doses, reduced doses and titration doses, for patients. Additionally or alternatively, a HCP and/or patients can interface with the computing device 510 through the display/UI 518 to input information about the patient, such as glucose measurements or observations about patient events such as hypoglycemia, that are stored as part of the patient records 16 and/or otherwise used by the dosing module. Insulin doses determined by the computing device 510 and information inputted into the computing device by the display/UI 518 may be displayed by the display and/or transmitted to other components of the system 500 such as for example to the drug delivery device 540 (e.g., for control of the delivery of the drug by the delivery device) and/or to the server 560 (e.g., for storage in the patent's records 16).

Glucose sensing device 520 illustratively includes any sensor adapted to measure a glucose level of a person with diabetes, such as a blood glucose monitor (BGM), a continuous glucose monitor (CGM), and/or a flash glucose monitor (FGM). The illustrated embodiments of the glucose sensor 520 include a processing circuit 522, a glucose sensor 524, and communication device 526. Processing circuit 522 may include any processing circuit that receives and processes data signals, and which outputs results in the form of one or more electrical signals as a result. Processing circuit 522 may include a processor (similar to processor 512), an Application Specific Integrated Circuit (ASIC), field-programmable gate arrays (FPGAs), digital signal processors (DSPs), hardwired logic, or combinations thereof. Glucose sensor 524 comprises any sensor capable of extracting and/or analyzing analyte (e.g., blood or interstitial fluid) from the body of the person with diabetes to measure and/or record the person's glucose levels. Communication device 526 allows glucose sensor 520 to communicate with other components of the system 500, such as for example computing device 510 and/or server 560 (e.g., to relay the measured glucose levels for use by the computing device and/or storage in the patient records 16).

Sensor 530 illustratively may include any sensor configured to measure at least one of a physiological or medical parameter of the subject (e.g., indications of hypoglycemia or hyperglycemia), a geographical or physical location of the person, and movement of the person, and to communicate the measured information to computing device 510 and/or server 560. Sensor 530 may be a wearable and/or portable sensor configured to be worn on, attached to, or carried by a person with diabetes. Examples of wearable sensors includes smartwatches (e.g., the Apple Watch®, a Fitbit®, and the like), heart-rate monitors, cardiac monitors, and the like. Sensor 530 may also be an implantable sensor implanted within the person's body. In yet other embodiments, sensor 530 may be neither wearable nor implantable, but may be configured to observe the person with diabetes. For example, sensor 530 may be a pressure and/or movement sensor placed on top of, underneath, or within the person's bed, and configured to record the times at which the person sleeps on the bed, as well as information regarding the quality of the person's sleep. Sensor 530 may also comprise a camera placed in the person's home, office, car, or other location that is configured to observe the person's behavior, presence, and/or appearance. The illustrated embodiments of sensor 530 includes a processing circuit 532, one or more sensor(s) 534 configured to measure the above-referenced information regarding the person, and communication device 536. Processing circuit 532 may include any of the possible types of processing circuits previously described. Communication device 536 allows sensor 530 to communicate with other components of the system 500, such as computing device 510 and server 560, via network 550.

Server 560 illustratively includes any computing device configured to receive information regarding a person with diabetes from components of system 500 such as the computing device 510 and/or the glucose sensing device 520 via network 550. The illustrated embodiments of server 560 include processing circuit 562, memory 564, and communication device 566. Processing circuit 562 may include any of the possible types of processing circuits previously described. Processing circuit 562 may execute software and/or firmware stored in memory 564 of server 560. The software/firmware code contains instructions that, when executed by processing circuit 562 to perform any or all of the functions described herein. Memory 564 may also be configured to store information regarding one or more persons with diabetes, such as biographical information and/or medical information (e.g., insulin dosing records, medical history, and the like). Information received from or sent to computing device 510 may also be stored in memory 564. Memory 564 may include any of the possible types of memory previously described. Communication device 566 allows server 560 to communicate with other components of the system 500 such as the computing device 510, glucose sensing device 520 and/or drug delivery device 540 via network 550.

In embodiments, for example, the memory 564 of the server 560 may include instructions for implementing any or all of the methods described above in connection with the dosing module 12. For example, the server 560 may provide a website or other functionality enabling a user of a computing device 510 to determine one or more loading doses, reduced doses or titration doses (e.g., by software as a service or SaaS). Additionally and alternatively, the memory 564 of the server 560 may store information of the types described above that are included in the patient records 16 and/or used by the dosing module 12. Insulin doses such as one or more loading doses, reduced doses and titration doses determined by the dosing module 12 may be stored in the memory 564 of the server 560, for example as elements of the patient records 16.

System 500 has been described herein as implementing detector 514 within processor 510. Detector 514 may be configured to detect one or more indications that the user missed an insulin bolus or needs a bolus. Detector 514 may be configured to detect such indications of missed insulin boluses using different sensitivity levels. Detector 514 may comprise software instructions and/or logic that is stored in memory 516 and which is executed by processor 512 to implement the functionality described herein. Detector 514 may take other forms in other embodiments. For example, the functionality performed by detector 514 may be implemented at least partially by dedicated hardware and/or firmware, such as a separate and dedicated processor and/or processing circuit. In some embodiments, the functionality performed by detector 514 may also be implemented wholly or partially on server 560, glucose sensing device 520, sensor 530, and/or drug delivery device 540.

Drug delivery device 540 may include any conventional or otherwise known device configured to deliver doses of insulin to a person with diabetes, including one or more loading doses, reduced doses and/or titration doses determined by the dosing module 12. Embodiments of drug delivery device 540 may also be configured to measure and/or record the time and amount of dose delivered, and/or to communicate this information to other components of the system 500, such as computing device 510 and sever 560 (e.g., for storage in the patient records 16 (as shown in FIG. 1)). Embodiments of the drug delivery device 540 may be operated in a manner generally as described herein by a patient, caregiver or health care provider to deliver insulin to a subject. The insulin delivered by device 540 may be formulated with one or more excipients. Drug delivery device 540 may be configured as a re-usable device that may be re-filled with insulin once its store of insulin is exhausted or may be configured as a disposable device that is designed to be discarded and replaced once its store of insulin is exhausted. Embodiments of drug delivery device 540 illustrated in FIG. 24 include processing circuit 542, dose detection sensor 544, and communication device 546. Processing circuit 542 may include any of the possible types of processing circuits previously described. Dose detection sensor 544 may include any suitable sensor for detecting and/or recording the time and amount of dose delivered. Communication device 546 allows drug delivery device 540 to communicate with other components of the system 500, such as for example the computing device 510 and/or the server 560 via the network 550 (e.g., to receive information about the doses to be delivered, and/or to provide information about delivered doses).

In embodiments, drug delivery device 540 is a manually-actuated or mechanically actuated device, such as an insulin pen, autoinjector, or a syringe, configured to enable a user to select or otherwise control the amount of insulin that is delivered. For example, one or more loading doses, reduced doses or titration doses determined in accordance with devices or methods described herein may be communicated to the user through the display/UI 518 of the computing device 510. The user of such a manually-actuated or mechanically-actuated drug delivery device 540 may configure the drug delivery device to deliver the dose communicated by the computing device 510.

In other embodiments, the drug delivery device 540 is a connected device. Examples of such connected delivery devices include an insulin pump or a connected insulin pen, such as a pen or autoinjector having integrated and/or attachable electronics to receive information about the predetermined doses (e.g., from a computing device 510 or server 560, and to automatically deliver the corresponding doses. Non-limiting examples of drug delivery devices 540 are disclosed in the following documents: (1) PCT Application No. PCT/US17/65251 filed Dec. 8, 2017 and entitled Medication Delivery Device with Sensing System, and (2) PCT Application No. PCT/US18/19156 filed Feb. 22, 2018 and entitled Dose Detection and Drug Identification for a Medication Delivery Device.

### SUMMARY

Exemplary aspects of the present disclosure include the following:
1. A method for operating a safety module of a computing system to determine a reduced medication dosage for management of a disease in a subject, the method comprising: receiving records of the subject relating to the disease;
determining a current medication activity level in the subject based on the subject's records;
determining a medication activity level reduction based on the subject's records;
determining a target medication activity level based on the current medication activity level and the medication activity level reduction;
determining a proposed medication dose based on the target medication activity level;
determining a first set of one or more predicted medication activity levels in the subject based on the proposed medication dose;
comparing the medication activity levels of the first set to a maximum medication activity level;
verifying the proposed medication dose as a recommended reduced dose if the predicted medication activity levels of the first set are less than or equal to the maximum medication activity level;
iteratively determining one or more updated proposed medication doses if one or more of the predicted medication activity levels of the first set is greater than the maximum medication activity level, including iteratively:
   determining the updated proposed medication dose by reducing the proposed medication dose or a previously determined updated proposed medication dose by a predetermined amount;
   determining an updated set of one or more predicted medication activity levels in the subject based on the updated proposed medication dose; and
   comparing the medication activity levels of the updated set to the maximum medication activity level, and
verifying the updated proposed medication dose as the recommended reduced dose if the associated one or more predicted medication activity levels of the updated set are less than or equal to the maximum medication activity level.
2. The method of aspect 1, wherein determining the medication activity level reduction comprises determining the medication activity level reduction based on a number of entries in the subject's records relating to the disease within a predetermined period of time of the medication dosage determination.
3 The method of any of aspects 1-2, wherein determining the medication activity level reduction comprises determining the medication activity level reduction based on one or both of (1) a number of measurements of the subject's physiological parameter representative of the disease within a predetermined period of time of the medication dosage determination or (2) the values of the measurements.
4. The method of any of aspects 1-3, wherein determining the medication activity level reduction comprises determining the medical activity level reduction based on one or both of (1) a number of the health care provider entries representative of the disease within a predetermined period of time of the medication dosage determination or (2) attributes of the health care provider entries.
5. The method of aspect 4, wherein determining the medication activity level reduction comprises determining the medication activity level reduction based on one or both of (1) a number of measurements of the subject's physiological parameter representative of the disease within a predetermined period of time of the medication dosage determination or (2) the values of the measurements.
6. The method of any of aspects 1-5, wherein determining the medication activity level reduction comprises:
determining types and numbers of reduction counts based on information in the subject's records within a predetermined period of time of the medication dosage determination;
determining safety concern levels based on the types and numbers of the reduction counts; and
determining the medication activity level reduction based on the determined safety concern levels.
7. The method of aspect 6, wherein determining the medication activity level reduction comprises determining the medication activity level reduction based on a number of measurements of the subject's physiological parameter representative of the disease within a predetermined period of time of the medication dosage determination.
8 The method of any of aspects 1-7, wherein the medication is configured for delivery to the subject periodically at delivery time periods, and wherein determining the first set of predicted medication activity levels and any updated sets of predicted medication activity levels includes determining medication activity levels over two or more delivery time periods.
9. The method of any of aspects 1-8, further comprising:
determining safety concern levels based on information in the subject's records within a predetermined period of time of the medication determination;
comparing the safety concern levels to predetermined criteria;
operating the safety module to determine the recommended reduced dose by the method of aspect 1 when the safety concern levels match a first criteria; and
operating a titration module of the computer system to determine a recommended titration dose by a method different than the method of aspect 1 when the safety concern levels match a second criteria that is different than the first criteria.
10. The method of aspect 9, wherein determining the safety concern levels comprises determining the safety concern levels based on one or both of (1) a number of measurements of the subject's physiological parameter representative of the disease within a predetermined period of time of the medication dosage determination or (2) the values of the measurements.
11. The method of aspect 9, wherein determining the safety concern levels comprises determining the safety concern levels based on one or both of (1) a number of the health care provider entries representative of the disease within a predetermined period of time of the medication dosage determination or (2) attributes of the health care provider entries.
12. The method of aspect 11, wherein determining the safety concern levels comprises determining the safety concern levels based on one or both of (1) a number of measurements of the subject's physiological parameter representative of the disease within a predetermined period of time of the medication dosage determination, or (2) the values of the measurements.
13. The method of aspect 9, wherein determining a recommended titration dose comprises:
determining a proposed titration dose;
determining a first set of one or more predicted medication activity levels in the subject based on the proposed titration dose;
comparing the medication activity levels of the first set to a maximum medication activity level,
verifying the proposed titration dose as a recommended titration dose if the predicted medication activity levels of the first set are less than or equal to the maximum medication activity level;
iteratively determining one or more updated proposed titration doses if one or more of the predicted medication activity levels of the first set is greater than the maximum medication activity level, including iteratively:
   determining the updated proposed titration dose by reducing the proposed titration dose or a previously determined updated proposed titration dose by a predetermined amount;
   determining an updated set of one or more predicted medication activity levels in the subject based on the updated proposed titration dose; and
   comparing the medication activity levels of the updated set to the maximum medication activity level; and
verifying the updated proposed titration dose as the recommended titration dose if the associated one or more predicted medication activity levels of the updated set are less than or equal to the maximum medication activity level.
14. The method of aspect 9, wherein operating the titration module of the computer system to determine the recommended titration dose comprises:
determining whether to determine the recommended titration dose by an explore module or an exploit module based on the subject's records;
determining the recommended titration dose by an explore module method when the explore module is determined; and
determining the recommended titration dose by an exploit module method when the exploit module is determined, wherein the exploit module method is different than the explore module method.
15. The method of aspect 14, wherein determining whether to determine the recommended titration dose by the explore module or the exploit module comprises:
determining whether a first previous recommended titration dose was determined by the explore module method or the exploit module method;
if the first previous recommended titration dose was determined by the explore module method, determining whether to transition to the exploit module method based on a comparison of information in the subject's records to a first set of transition criteria; and
if the first previous recommended titration dose was determined by the exploit module method, determining whether to transition to the explore module method based on a comparison of information in the subject's records to a second set of transition criteria, wherein the second set of transition criteria is different than the first set of transition criteria.
16. The method of aspect 15, wherein determining whether to transition to the exploit module method if the first previous recommended titration dose was determined by the explore module method comprises:
comparing information in the subject's records during a second previous dose period to a first subset of the first set of transition criteria;
not transitioning, and determining the titration dose by the explore module, if the information in the subject's records during the second previous dose period do not match the first subset of the first set of transition criteria;
comparing information in the subject's records during a first previous dose period to a second subset of the first set of transition criteria if the information in the subjects records during the second previous dose period to match the first subset of the first set of transition criteria;
not transitioning, and determining the titration dose by the explore module, if the information in the subject's records during the first previous dose period do not match the second subset of the first set of transition criteria; and
transitioning, and determining the titration dose by the exploit module, if the information in the subject's records during the first previous dose period matches the second subset of the first set of transition criteria.
17. The method of aspect 16, wherein the criteria of one or more of the first and second subsets of the first set of transition criteria comprise one or both of (1) a predetermined number of measurements of the subject's physiological parameter representative of the disease or (2) predetermined values of the measurements.
18. The method of aspect 16, wherein the criteria of one or more of the first and second subsets of the first set of transition criteria comprise one or both of (1) a predetermined number of the health care provider entries representative of the disease or (2) predetermined attributes of the health care provider entries.
19. The method of aspect 18, wherein the criteria of the first and second subsets of the first set of transition criteria comprise one or both of (1) a predetermined number of measurements of the subject's physiological parameter representative of the disease or (2) predetermined values of the measurements.
20. The method of aspect 14, wherein determining whether to transition to the explore module method if the first previous dose was determined by exploit module method comprises:
comparing information in the subject's records during a first previous dose period to a first subset of the second set of transition criteria;
transitioning, and determining the titration dose by the explore module, if the information in the subject's records during the first previous dose period do not match the first subset of the second set of transition criteria;
comparing information in the subject's records during a second previous dose period to a second subset of the second set of transition criteria if the information in the subject's records during the first previous dose period match the first subset of the second set of transition criteria;
not transitioning, and determining the titration dose by the exploit module, if the information in the subject's records during the second previous dose period do not match the second subset of the second set of transition criteria;
comparing information in the subject's records during the first previous dose period to a third subset of the second set of transition criteria if the information in the subject's records during the second previous dose period matches the second subset of the second set of transition criteria;
not transitioning, and determining the titration dose by the exploit module, if the information in the subject's records during the first previous dose period do not match the third subset of the second set of transition criteria, and
transitioning, and determining the titration dose by the explore module, if the information in the subject's records during the first previous dose period matches the third subset of the second set of transition criteria.
21. The method of aspect 20, wherein the criteria of one or more of the first, second and third subsets of the second set of transition criteria comprise one or both of (1) a predetermined number of measurements of the subject's physiological parameter representative of the disease or (2) predetermined values of the measurements.
22. The method of aspect 20, wherein the criteria of one or more of the first, second and third subsets of the second set of transition criteria comprise one or both of (1) a predetermined number of the health care provider entries representative of the disease or (2) predetermined attributes of the health care provider entries.
23. The method of aspect 22, wherein the criteria of one or more of the first, second and third subsets of the second set of transition criteria comprise one or both of (1) a predetermined number of measurements of the subject's physiological parameter representative of the disease or (2) predetermined values of the measurements.
24. The method of aspect 14, wherein determining the recommended titration dose by the explore module comprises determining the recommended titration dose using a closed loop algorithm.
25. The method of aspect 24, wherein determining the recommended titration dose comprises:
determining one or more error values based on a target value of a physiological parameter representative of the disease and one or more measured values of the physiological parameter of the subject;
determining one or more dose adjust components based on the one or more error values;
determining a dose adjustment based on the one or more dose adjust components, and
determining the recommended titration dose based on the dose adjustment and a first previously recommended dose.
26. The method of aspect 25, wherein the closed loop algorithm is a proportional—derivative algorithm, and wherein:
determining the one or more dose adjust components comprises:
   determining a proportional adjust component based on (1) one or more of the one or more error values, and (2) one or more measured values of one or more physiological parameters representative of the disease in the subject; and
   determining a derivative adjust component based on one or more of the one or more error values; and
determining the dose adjustment comprises determining the dose adjustment based on the proportional adjust component and the derivative adjust component.
27. The method of aspect 26, wherein:
determining the proportional adjust component comprises determining the proportional adjust component based on a proportional parameter; and
determining the derivative adjust component comprises determining the derivative adjust component based on a derivative parameter.
28. The method of aspect 27, wherein:
determining the one or more error values comprises:
   determining a current error value based the target value of the physiological parameter and one or more measured values of the physiological parameter during a first previous dose period;
   determining a previous error value based on the target value of the physiological parameter and one or more measured values of the physiological parameter during at least a second previous dose period, wherein the second previous dose period is a period before the first previous dose period; and
   determining a delta error based on the current error value and the previous error value;
determining the proportional adjust component comprises determining the proportional adjust component based on the proportional parameter and the current error value; and
determining the derivative adjust component comprises determining the derivative adjust component based on the derivative parameter and the delta error value.
29. The method of aspect 28, wherein determining the proportional parameter comprises determining the proportional parameter based on an initial value of the proportional parameter and the current error.
30. The method of aspect 29, comprising:
   determining whether the recommended titration dose is a boost dose; and
   adjusting the proportional parameter by a boost factor if a boost dose is determined.
31. The method of aspect 30, wherein determining whether the recommended titration dose is a boost dose comprises determining whether the recommended titration dose is a boost dose based on one or more criteria comprising (1) a number of measurements of the subject's physiological parameter representative of the disease, (2) values of the measurements of the subjects physiological parameter, (3) a number of the health care provider entries representative of the disease, (4) attributes of the health care provider entries representative of the disease, or (5) whether a boost dose was recommended within a predetermined number of previous dose periods.
32. The method of aspect 31, wherein the boost factor is based on a number of measured values of the physiological parameter.
33. The method of aspect 31, wherein the boost factor is based on a number of measured values of the physiological parameter.
34. The method of aspect 14, wherein determining the recommended titration dose by the exploit module comprises:
determining whether the first previous dose was determined by the explore module or the exploit module;
if the first previous dose was determined by the exploit module, determining the recommended titration dose based on the first previous dose; and
if the first previous dose was determined by the explore module, determining the recommended titration dose based on a current medication activity level in the subject based on the subject's records.
35. The method of aspect 34, comprising verifying and optionally adjusting the recommended titration dose based on a first criteria and predicted medication activity levels in the subject.
36. The method of aspect 35, comprising verifying and optionally adjusting the recommended titration dose based on a second criteria and relating to predicted medication activity levels in the subject, wherein the second criteria is different than the first criteria.

Exemplary aspects of the present disclosure further include the following:
1. A system comprising:
   a computing system programmed to calculate a final insulin dose using an operation comprising:
      determining a current insulin activity level;
      determining an insulin activity reduction amount based, at least in part, on a glucose level;
      calculating a target insulin activity level based, at least in part, on the current insulin activity level and the insulin activity reduction amount;
      determining a proposed insulin dose based, at least in part, on the target insulin activity level.
2. The system of aspect 1, wherein the determining the insulin activity reduction amount is further based, at least in part, on an attribute relating to one or more hypoglycemia events.
3. The system of aspect 2, wherein the attribute comprises a number of hypoglycemia events.
4. The system of aspect 2, wherein the insulin activity reduction amount increases with an increased number of hypoglycemia events.
5. The system of aspect 2, wherein the attribute comprises a time of day when the one or more hypoglycemia events occurred.
6 The system of aspect 5, wherein the insulin activity reduction amount is a set percentage when the attribute is a nocturnal hypoglycemia event.
7. The system of aspect 1, wherein the determining the insulin activity reduction amount is further based, at least in part, on a number of available glucose measurements within a predetermined time period.
8. The system of aspect 1, wherein the determining the insulin activity reduction amount is further based, at least in part, on a table correlating different insulin activity reduction amounts with different glucose levels.
9. The system of aspect 1, wherein the determining the insulin activity reduction amount is further based, at least in part, on an equation with different reductions based on a median glucose level.
10. The system of aspect 9, wherein the insulin activity reduction amount decreases as the median glucose level increases.
11. A method comprising:
   determining a current insulin activity level;
   determining an insulin activity reduction amount based, at least in part, on a glucose level;
   calculating a target insulin activity level based, at least in part, on the current insulin activity level and the insulin activity reduction amount;
   determining a proposed insulin dose based, at least in part, on the target insulin activity level.
12. The system of aspect 11, wherein the determining the insulin activity reduction amount is further based, at least in part, on an attribute relating to one or more hypoglycemia events
13. The method of aspect 12, wherein the attribute comprises a number of hypoglycemia events.
14. The method of aspect 12, wherein the insulin activity reduction amount increases with an increased number of hypoglycemia events.
15. The method of aspect 12, wherein the attribute comprises a time of day when the one or more hypoglycemia events occurred.
16. The method of aspect 15, wherein the insulin activity reduction amount is a set percentage when the attribute is a nocturnal hypoglycemia event.
17. The method of aspect 11, wherein the determining the insulin activity reduction amount is further based, at least in part, on a number of available glucose measurements within a predetermined time period.
18. The method of aspect 11, wherein the determining the insulin activity reduction amount is further based, at least in part, on a table correlating different insulin activity reduction amounts with different glucose levels.
19. The method of aspect 11, wherein the determining the insulin activity reduction amount is further based, at least in part, on an equation with different reductions based on a median glucose level.
20. The method of aspect 19, wherein the insulin activity reduction amount decreases as the median glucose level increases.
21. The method of aspect 11, wherein the operation further comprises:
   iteratively adjusting the proposed insulin dose until a predicted insulin activity level is less than a predetermined maximum threshold; and
   determining a final insulin dose after the iteratively adjusting.
22. The method of aspect 21, wherein the proposed insulin dose is iteratively reduced until the proposed insulin dose results in the predicted insulin activity level being less than the predetermined maximum threshold.
23. A computer program product comprising instructions to cause one or more processors to carry out the steps of the method of aspects 11-22.
24. A computer-readable medium having stored thereon the computer program product of aspect 23.
25. A computer comprising the computer-readable medium of aspect 24.

Exemplary aspects of the present disclosure further include the following:
1. A system comprising:
   a computing system programmed to determine whether to use a first code module or a second code module to calculate an insulin dose, wherein the first code module is programmed to calculate the insulin dose based on a first set of criteria, wherein the second code module is programmed to calculate the insulin dose based on a second set of criteria different from the first criteria, wherein determining whether to use the first code module or the second code module comprises an operation comprising:
   determining whether the first code module or the second code module was used to calculate a prior insulin dose,
   if the first code module was used to calculate the prior insulin dose, use the second code module to calculate the insulin dose after (1) determining that safety event data from a prior dose period is less than a safety threshold and (2) determining that a glucose value from the prior dose period is below a first glucose threshold, and
   if the second code module was used to calculate the prior insulin dose, use the first code module to calculate the insulin dose by determining that either (1) safety event data from the prior dose period is greater than a safety threshold or (2) that the glucose value from the prior dose period is above a second glucose threshold
2. The system of aspect 1, wherein the first code module is programmed to operate a closed loop algorithm.
3. The system of aspect 2, wherein the first code module is programmed to:
   determine an error value based on a target glucose value and a measured glucose value;
   determine an insulin dose adjustment based on the error value; and
   determine the insulin dose based on the insulin dose adjustment and a previously recommended insulin dose.
4. The system of aspect 3, wherein the closed loop algorithm is a proportional—derivative algorithm, and wherein determining the dose adjustment comprises:
   determine a proportional adjust component based on (1) the error value and (2) the measured glucose value, and
   determine a derivative adjust component based on the error value.
5. The system of aspect 4, wherein the proportional adjust component is further based on weight of a subject.
6. The system of aspect 3, wherein the error value is determined by:
   determining a current error value based the target glucose value and the measured glucose value during a first previous dose period,
   determining a previous error value based on the target glucose value and the measured glucose value during a second previous dose period, wherein the second previous dose period is a period before the first previous dose period, and
   determining a delta error based on the current error value and the previous error value.
7. The system of aspect 6, wherein the proportional adjust component is based on a proportional parameter and the current error value, wherein the derivative adjust component based on a derivative parameter and the delta error value.
8. The system of aspect 7, wherein the proportional parameter is based on an initial value of the proportional parameter and the current error.
9. The system of aspect 3, wherein determining the error value is based on a regression formula.
10. The system of aspect 9, wherein the regression formula is utilized only if a minimum number of measured glucose values are available.
11. A method for determining whether to use a first code module or a second code module to calculate an insulin dose, wherein the first code module is programmed to calculate the insulin dose based on a first set of criteria, wherein the second code module is programmed to calculate the insulin dose based on a second set of criteria different from the first criteria, the method comprising:
   determining whether the first code module or the second code module was used to calculate a prior insulin dose,
   if the first code module was used to calculate the prior insulin dose, using the second code module to calculate the insulin dose after (1) determining that safety event data from a prior dose period is less than a safety threshold and (2) determining that a glucose value from the prior dose period is below a first glucose threshold, and
   if the second code module was used to calculate the prior insulin dose, using the first code module to calculate the insulin dose by determining that either (1) safety event data from the prior dose period is greater than a safety threshold or (2) that the glucose value from the prior dose period is above a second glucose threshold.
12. The system of aspect 11, wherein the first code module is programmed to operate a closed loop algorithm.
13. The method of aspect 12, wherein the first code module is programmed to:
   determine an error value based on a target glucose value and a measured glucose value;
   determine an insulin dose adjustment based on the error value; and
   determine the insulin dose based on the insulin dose adjustment and a previously recommended insulin dose.
14. The method of aspect 13, wherein the closed loop algorithm is a proportional—derivative algorithm, and wherein determining the dose adjustment comprises:
   determine a proportional adjust component based on (1) the error value and (2) the measured glucose value, and
   determine a derivative adjust component based on the error value.
15. The method of aspect 14, wherein the proportional adjust component is further based on weight of a subject.
16. The method of aspect 13, wherein the error value is determined by:
   determining a current error value based the target glucose value and the measured glucose value during a first previous dose period,
   determining a previous error value based on the target glucose value and the measured glucose value during a second previous dose period, wherein the second previous dose period is a period before the first previous dose period, and
   determining a delta error based on the current error value and the previous error value.
17. The method of aspect 16, wherein the proportional adjust component is based on a proportional parameter and the current error value, wherein the derivative adjust component based on a derivative parameter and the delta error value.
18. The system of aspect 17, wherein the proportional parameter is based on an initial value of the proportional parameter and the current error.
19. The method of aspect 13, wherein determining the error value is based on a regression formula.
20. The method of aspect 19, wherein the regression formula is utilized only if a minimum number of measured glucose values are available.
21. The method of aspect 11, wherein the second code module is programmed to calculate the insulin dose such that the insulin dose is equal to a previous insulin dose.
22. The method of aspect 11, wherein the first code module is programmed to operate a closed loop algorithm, wherein the second code module does not include a closed loop algorithm.
23. A computer program product comprising instructions to cause one or more processors to carry out the steps of the method of aspects 11-22.
24. A computer-readable medium having stored thereon the computer program product of aspect 23.
25. A computer comprising the computer-readable medium of aspect 24.

Exemplary aspects of the present disclosure further include the following:
1. A system comprising:
   a computing system programmed to calculate a boost dose using an operation comprising:
      determining that the boost dose should be calculated based on:
         determining that a minimum amount of time has passed since a prior boost dose,
         determining that a hypoglycemia event has occurred within a time period, and
         determining that a minimum number of glucose measurements from a prior period of time are available, and
      calculating the boost dose based on the glucose measurements.
2. The system of aspect 1, wherein an amount of the boost dose depends on a number of available glucose measurements.
3. The system of aspect 2, wherein the amount of the boost dose is larger with a larger number of the available glucose measurements.
4. The system of aspect 2, wherein the computing system includes a first code module to determine a final insulin dose, wherein the first code module is programmed to operate a proportional—derivative algorithm, wherein the boost dose increases a proportional adjustment component of the proportional—derivative algorithm.
5. The system of aspect 2, wherein the operation further comprises:
   iteratively adjusting the boost dose until a predicted insulin activity level associated with the boost dose is less than a predetermined maximum threshold; and
   determining a final boost dose after the iteratively adjusting.
6. A method of providing glycemic control in a subject in need thereof having diabetes, the method comprising:
   administering to the subject an initial dose of weekly basal insulin;
   determining that a boost dose should be calculated based on:
      determining that a minimum amount of time has passed since a prior boost dose,
      determining that a hypoglycemia event has occurred within a time period, and
      determining that a minimum number of glucose measurements from a prior period of time are available,
   calculating the boost dose based on the glucose measurements, and
   administering to the subject a maintenance dose of basal insulin based, at least in part, on the boost dose calculated.
7. The method of aspect 6, wherein an amount of the boost dose depends on a number of available glucose measurements.
8 The method of aspect 6, wherein the amount of the boost dose is larger with a larger number of the available glucose measurements.
9 The method of aspect 6, wherein the maintenance dose is calculated by a proportional—derivative algorithm, wherein the boost dose increases a proportional adjustment component of the proportional—derivative algorithm.
10. The method of aspect 6, further comprising:
   iteratively adjusting the boost dose until a predicted insulin activity level associated with the boost dose is less than a predetermined maximum threshold; and
   determining a final boost dose after the iteratively adjusting.
11. A method comprising:
   determining that a boost dose should be calculated based on:
      determining that a minimum amount of time has passed since a prior boost dose,
      determining that a hypoglycemia event has occurred within a time period, and
      determining that a minimum number of glucose measurements from a prior period of time are available, and
   calculating the boost dose based on the glucose measurements.
12. The method of aspect 11, wherein an amount of the boost dose depends on a number of available glucose measurements.
13. The method of aspect 12, wherein the amount of the boost dose is larger with a larger number of the available glucose measurements.
14. The method of aspect 11, further comprising:
   iteratively adjusting the boost dose until a predicted insulin activity level associated with the boost dose is less than a predetermined maximum threshold; and
   determining a final boost dose after the iteratively adjusting.
15. The method of aspect 11, wherein the minimum amount of time includes at least two prior dose periods.
16. The method of aspect 11, wherein the determining that the boost dose should be calculated is further based on a lowest of the glucose measurements being greater than a minimum threshold.
17. The method of aspect 16, wherein the determining that the boost dose should be calculated is further based on a median of the glucose measurements being greater than a median threshold.
18. The method of aspect 11, wherein the boost dose increases a proportional adjustment component of the proportional—derivative algorithm.
19. The method of aspect 18, wherein the boost dose is a multiplying factor applied to the proportional adjustment component.
20. The method of aspect 18, further comprising:
   calculating a final insulin dose using the proportional—derivative algorithm.
21. The method of aspect 20, wherein the proportional—derivative algorithm includes:
   determine an error value based on a target glucose value and a measured glucose value;
   determine an insulin dose adjustment based on the error value; and
   determine the insulin dose based on the insulin dose adjustment and a previously recommended insulin dose.
22 The method of aspect 21, wherein determining the dose adjustment comprises:
   determine the proportional adjust component based on (1) the error value and (2) the measured glucose value and multiplying by the boost dose, and
   determine a derivative adjust component based on the error value.
23. A computer program product comprising instructions to cause one or more processors to carry out the steps of the method of aspects 11-22
24. A computer-readable medium having stored thereon the computer program product of aspect 23.
25. A computer comprising the computer-readable medium of aspect 24.

Various alternatives and modifications may be devised by those skilled in the art without departing from the present disclosure. For example, although the disclosure uses a model-based controller to ultimately determine and deliver an appropriate amount of insulin to a subject, features of the disclosure can apply to other types of control algorithms (e.g., proportional-integral derivative (PID) control algorithm, a fuzzy logic control algorithm, and the like).

Accordingly, the present disclosure is intended to embrace all such alternatives, modifications and variances. Additionally, while several embodiments of the present disclosure have been illustrated in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments.

1. A system comprising:
a computing system programmed to calculate a final insulin dose using an operation comprising:
calculating a proposed insulin dose;
iteratively adjusting the proposed insulin dose until a predicted first level is greater than a first threshold;
iteratively adjusting the proposed insulin dose until a predicted second level is less than a predetermined maximum threshold; and
determining the final insulin dose after the iteratively adjusting steps.

2. The system of claim 1, wherein the predicted first level is a median insulin activity level, wherein the first threshold is a subject's median insulin activity level.

3. The system of claim 2, wherein the proposed insulin dose is iteratively increased until the proposed insulin dose results in the predicted median insulin activity level being greater than the subject's median insulin activity level.

4. The system of claim 1, wherein the predicted second level is a maximum insulin activity level, wherein the second threshold is a predetermined maximum threshold.

5. The system of claim 4, wherein the proposed insulin dose is iteratively decreased until the proposed insulin dose results in the predicted maximum insulin activity level being less than a predetermined maximum threshold.

6. The system of claim 1, wherein the predicted first level is a median insulin activity level, wherein the first threshold is a subject's median insulin activity level, wherein the predicted second level is a maximum insulin activity level, wherein the second threshold is a predetermined maximum threshold.

7. The system of claim 1, wherein the proposed insulin dose is based, at least in part, on a target insulin activity level.

8. The system of claim 7, wherein the target insulin activity level is based, at least in part, on a current insulin activity level and an insulin activity level reduction.

9. The system of claim 8, wherein the current insulin activity level and the insulin activity level reduction are based, at least in part, on a subject's records.

10. The system of claim 8, wherein the insulin activity level reduction is based, at least in part, on a number of entries in the subject's records relating to a disease within a predetermined period of time of calculating the proposed insulin dose.

11. A method comprising:
calculating a proposed insulin dose;
iteratively adjusting the proposed insulin dose until a predicted first level is greater than a first threshold;
iteratively adjusting the proposed insulin dose until a predicted second level is less than a predetermined maximum threshold; and
determining a final insulin dose after the iteratively adjusting steps.

12. The system of claim 11, wherein the predicted first level is a predicted median insulin activity level, wherein the first threshold is a subject's median insulin activity level.

13. The method of claim 12, wherein the proposed insulin dose is iteratively increased until the proposed insulin dose results in the predicted median insulin activity level being greater than the subject's median insulin activity level.

14. The method of claim 13, wherein the proposed insulin dose is iteratively increased based, at least in part, on median fasting blood glucose level.

15. The method of claim 12, wherein the predicted median insulin activity level is based, at least in part, on solving differential equations.

16. The method of claim 11, wherein the predicted second level is a predicted maximum insulin activity level, wherein the second threshold is a predetermined maximum threshold.

17. The method of claim 16, wherein the proposed insulin dose is iteratively decreased until the proposed insulin dose results in the predicted maximum insulin activity level being less than a predetermined maximum threshold.

18. The method of claim 17, wherein the proposed insulin dose is iteratively decreased based, at least in part, on median fasting blood glucose level.

19. The method of claim 16, wherein the predicted maximum insulin activity level is based, at least in part, on solving differential equations.

20. The method of claim 11, wherein the predicted first level is a median insulin activity level, wherein the first threshold is a subject's median insulin activity level, wherein the predicted second level is a maximum insulin activity level, wherein the second threshold is a predetermined maximum threshold.

21. The method of claim 11, wherein the proposed insulin dose is based, at least in part, on a target insulin activity level, wherein the target insulin activity level is based, at least in part, on a current insulin activity level and an insulin activity level reduction.

22.-25. (canceled)
